(19) United States
(12) Patent Application Publication
(10) Pub. No.: US 2026/0083641 A1
(43) Pub. Date: Mar. 26, 2026

(54) COMPOSITIONS COMPRISING COLLAGENASE AND USES THEREOF IN ORTHODONTIC PROCEDURES

(71) Applicant: RAMOT AT TEL AVIV UNIVERSITY LTD., Tel Aviv (IL)

(72) Inventors: Maayan GAL, Tel Aviv (IL); Orel COHEN, Tel Aviv (IL); Diva LUGASSY, Tel Aviv (IL); Itzhak BINDERMAN, Tel Aviv (IL); Evgeny WEINBERG, Tel Aviv (IL)

(21) Appl. No.: 19/404,309

(22) Filed: Dec. 1, 2025

Related U.S. Application Data
(63) Continuation of application No. PCT/IL2024/050573, filed on Jun. 10, 2024.
(60) Provisional application No. 63/472,669, filed on Jun. 13, 2023.

(51) Int. Cl. A61K 6/50; A61K 6/69

(52) U.S. Cl. CPC A61K 6/50; A61K 6/69; C12Y 304/24003

(57) ABSTRACT

Provided herein compositions including collagenase, and uses thereof in orthodontic procedures, wherein the compositions may include recombinant collagenase and/or modified forms of recombinant collagenase having amino acid(s) truncation or substitutions.

### CROSS REFERENCE TO RELATED APPLICATIONS

This application is a Bypass Continuation of PCT Patent Application No. PCT/IL2024/050573 having International filing date of Jun. 10, 2024, which claims the benefit of priority of U.S. Provisional Patent Application No. 63/472,669, filed Jun. 13, 2023, the contents of which are all incorporated herein by reference in their entirety.

### REFERENCE TO AN ELECTRONIC SEQUENCE LISTING

The contents of the electronic sequence listing (RMT_027-PCT.xml; Size: 18,107 bytes; and Date of Creation: Jun. 10, 2024) is herein incorporated by reference in its entirety.

### FIELD OF THE INVENTION

The present invention relates to compositions including collagenase and uses thereof in orthodontic procedures, wherein the compositions can include recombinant forms of a wild type (WT) collagenase, truncated collagenase (ColG), and/or modified forms of the truncated collagenases having amino acid(s) substitutions.

### BACKGROUND OF THE INVENTION

Acceleration of orthodontic tooth movement (OTM) and prevention of post-treatment relapse are of high importance in the field of orthodontics. Currently, most approaches reported to accelerate orthodontic tooth movement involve surgical or mechanical procedures. Likewise, post-orthodontic relapse is managed using fixed or removable appliances, which are difficult for use and may involve various complications.

Surgical approaches for accelerating orthodontic tooth movement (OTM) are associated with significant invasiveness, exposing the patient to additional stress and postoperative pain. For example, alveolar corticotomies (ACS) may be performed shortly before applying orthodontic forces. The procedure included raising a mucoperiosteal flap to perform corticotomy that enhances alveolar bone remodeling allowing acceleration of tooth movement and increased post-orthodontic stability. In other procedures, fiberotomy is used, including surgical detachment of the marginal gingiva from the root surface, triggering the remodeling of periodontal tissues and accelerating tooth movement and reducing post-treatment relapse.

Orthodontic relapse is defined as the post-treatment changes induced by the withdrawal of orthodontic forces and biologically is the reversed action of orthodontic tooth movement, where the tension side of the PDL can be considered the pressure side during relapse. Relapse is necessitating the clinician to splint the post-treated dentition by either a fixed or removable retainer. Among the complications of fixed retainers are detachments, fractures, unexpected tooth movements, and difficulty in maintaining correct oral hygiene. Removable retainers were found to be associated with discomfort, with those using the Hawley retainer having higher levels of embarrassment in terms of speech and esthetics. In addition, the long-term stability of mandibular anterior teeth after premolar extractions and edgewise orthodontic therapy followed by retention has shown that the success at maintaining proper mandibular anterior alignment is less than 30%.

Collagen is the main structural protein in the extracellular matrix (ECM) in various connective tissues. Collagen is made of amino acids forming a triple helix of elongated fibril, also termed a collagen helix. Several types of Collagen are known, including Fibrillar (Type I, II, III, V, XI) and Non-fibrillar. Collagen can be enzymatically degraded by enzymes, such as Collagenases, which break the peptide bonds in collagen.

Collagenase is a matrix metalloproteinase which can degrade extracellular collagen.

Collagenases have been used clinically with various applications such as: wound debridement and healing and removing scar tissues.

Thus, there is a need in the art for compositions including collagenase, for use in various orthodontic procedures in a robust, safe, efficient, cost-effective manner, while exhibiting reduced side effects and enhanced results as compared to surgical or mechanical procedures.

### SUMMARY OF THE INVENTION

According to some embodiments, there are provided compositions including collagenase (various optional forms thereof) and methods of using the same for enhancing various orthodontic procedures, including, accelerated orthodontic tooth movement, post-orthodontic relapse prevention and teeth alignment. In some embodiments, the compositions and methods disclosed herein can advantageously shorten the treatment time of active orthodontic treatment, prevent or reduce post orthodontic relapse in the retention phase, may be used as an alternative to surgically facilitated orthodontic therapy, may be used as an alternative approach to a permanent orthodontic retainer, may be used in addition to (and in synergy to) clear alignment treatment or other orthodontic treatments, and the like, or any combinations thereof. Each possibility is a separate embodiment.

In some embodiments, the collagenase may include recombinant collagenase polypeptides, full-length wild type (WT) collagenase (for example, but not necessarily, SEQ ID NO: 8), N-terminally truncated collagenase (for example, but not necessarily, SEQ ID NO: 1 and 9) and/or N-terminally truncated modified collagenase polypeptide(s) which include one or more point mutation(s) (for example, but not necessarily, SEQ ID NOs: 2-7) compared to the un-modified collagenases (truncated or WT), or homologs thereof. Each possibility is a separate embodiment.

Advantageously, in some embodiments, the truncated modified collagenase comprises at least one mutation that enhances its thermo-stability with respect to the N-terminally truncated un-modified collagenase or the full-length WT collagenase by at least 1.0° C.

Further advantageous, in some embodiments, the administration of recombinant collagenase polypeptides and compositions comprising the same, is performed into the connective tissue of the laminal propria (marginal gingiva).

According to some aspects, there is provided a recombinant collagenase polypeptide, or a composition comprising the same, for use in orthodontic procedures in a subject in need thereof. Each possibility is a separate embodiment.

According to some embodiments, the orthodontic procedures comprise one or more procedures selected from accelerated orthodontic tooth movement (OTM), post-orthodontic relapse prevention and teeth alignment, or any combination thereof.

In some embodiments, the recombinant collagenase comprises one or more collagenases selected from: a full-length WT collagenase, N-terminally truncated un-modified collagenase, and N-terminally truncated modified collagenase, or any combination thereof. Each possibility is a separate embodiment.

In some embodiments, the N-terminal truncation comprises truncation of the first 118 amino acids or less, with respect to the full-length WT collagenase. Each possibility is a separate embodiment.

In some embodiments, the truncated modified recombinant collagenase differs from the wild-type collagenase or from the truncated un-modified recombinant collagenase by at least one amino acids substitution or deletion selected from a mutation(s) listed in Table 2; and wherein the at least one mutation enhances its thermo-stability with respect to the N-terminally truncated un-modified collagenase or the full-length WT collagenase by at least 1.0° C.; and wherein the thermo-stability is calculated as a midpoint of temperature inactivation (T50). Each possibility is a separate embodiment.

In some embodiments, the thermo-stability of the N-terminally truncated modified collagenase is characterized by a midpoint of temperature inactivation (T50) of at least about 54° C.

In some embodiments, the recombinant collagenase comprises an amino acid sequence having at least about 65% sequence identity to an amino acid sequence of a reference collagenase; wherein the reference collagenase comprises one or more collagenases selected from: a full-length WT collagenase having amino acid sequence as denoted by SED ID NO: 8, an N-terminally truncated collagenase having amino acid sequence as denoted by SED ID NO: 1 or SED ID NO: 9, and N-terminally truncated modified collagenase having amino acid sequence as denoted by any one of SED ID NOs: 2-7; or any combination thereof. Each possibility is a separate embodiment.

In some embodiments, the recombinant collagenase comprises an amino acid sequence having at least about 65% sequence identity to an amino acid sequence of the N-terminally truncated reference collagenase having amino acid sequence as denoted by SED ID NO: 1 or SED ID NO: 9. Each possibility is a separate embodiment.

In some embodiments, the collagenase originates from Clostridium H (histolyticum) and/or Clostridium T (Tetani). Each possibility is a separate embodiment. Each possibility is a separate embodiment.

In some embodiments, the collagenase originates from Clostridium H (histolyticum).

In some embodiments, the recombinant collagenase polypeptide comprises an amino acid sequence as denoted by SEQ ID NO: 1 and/or SEQ ID NO: 9. Each possibility is a separate embodiment.

In some embodiments, the recombinant collagenase polypeptide comprises an amino acid sequence as denoted by any one of SED ID NO: 2, SED ID NO: 3, SED ID NO: 4, SED ID NO: 5, SED ID NO: 6, SED ID NO: 7, or any combination thereof. Each possibility is a separate embodiment.

In some embodiments, the recombinant collagenase polypeptide comprises an amino acid sequence as denoted by SED ID NO: 2 and/or SED ID NO: 5. Each possibility is a separate embodiment.

In some embodiments, the recombinant collagenase polypeptide comprises an amino acid sequence as denoted by SEQ ID NO: 8.

In some embodiments, the recombinant collagenase polypeptide or the composition comprising the same are administered locally. Each possibility is a separate embodiment.

In some embodiments, the recombinant collagenase polypeptide or the composition comprising the same are administered locally to a connective tissue of marginal gingiva (lamina propria). Each possibility is a separate embodiment.

In some embodiments, the administration is by local injection.

In some embodiments, the recombinant collagenase polypeptide or the composition comprising the same are configured to affect gingival fibers, thereby providing enzymatic fiberotomy. Each possibility is a separate embodiment.

In some embodiments, the recombinant collagenase polypeptide is administered at an effective amount of at least about 1.2 mg/ml. In some embodiments, the recombinant collagenase polypeptide is administered at an effective amount of at least about 4 mg/ml.

In some embodiments, the orthodontic procedure utilizing the recombinant collagenase polypeptide or the composition comprising the same, provides equal or enhanced effect on tooth movement (mm), compared to same orthodontic procedure utilizing surgical fiberotomy; and wherein the orthodontic procedure comprises accelerated orthodontic tooth movement (OTM). Each possibility is a separate embodiment.

In some embodiments, the orthodontic procedure utilizing the recombinant collagenase polypeptide or the composition comprising the same, provides equal or improved reduction in relapse compared to relapse after same orthodontic procedure utilizing surgical fiberotomy; and wherein the orthodontic procedure comprises prevention of post-orthodontic relapse. Each possibility is a separate embodiment.

According to some aspects, there is provided a method of performing an orthodontic procedure in a subject in need thereof, the method comprising administering to the subject in need thereof an effective amount of a recombinant collagenase polypeptide, or a composition comprising the same. Each possibility is a separate embodiment.

According to some embodiments, the orthodontic procedure comprises one or more procedures selected from accelerated orthodontic tooth movement (OTM), post-orthodontic relapse prevention and teeth alignment, or any combination thereof. Each possibility is a separate embodiment.

In some embodiments, the recombinant collagenase comprises one or more collagenases selected from: a full-length WT collagenase, N-terminally truncated un-modified collagenase, and N-terminally truncated modified collagenase, or any combination thereof. Each possibility is a separate embodiment.

In some embodiments, the N-terminal truncation comprises truncation of at least 118 amino acid residues, with respect to the corresponding full-length WT collagenase. Each possibility is a separate embodiment.

In some embodiments, the truncated modified recombinant collagenase differs from the wild-type collagenase or from the truncated un-modified recombinant collagenase by at least one amino acids substitution or deletion selected from a mutation(s) listed in Table 2; and wherein the at least one mutation enhances its thermo-stability with respect to the N-terminally truncated un-modified collagenase or the full-length WT collagenase by at least 1.0° C.; and wherein the thermo-stability is calculated as a midpoint of temperature inactivation (T₅₀). Each possibility is a separate embodiment.

In some embodiments, the thermo-stability of the N-terminally truncated modified collagenase is characterized by a midpoint of temperature inactivation (T₅₀) of at least about 54° C.

In some embodiments, the recombinant collagenase comprises an amino acid sequence having at least about 65% sequence identity to an amino acid sequence of a reference collagenase; wherein the reference collagenase comprises one or more collagenases selected from: a full-length WT collagenase having amino acid sequence as denoted by SED ID NO: 8, an N-terminally truncated collagenase having amino acid sequence as denoted by SED ID NO: 1 or SED ID NO: 9, and N-terminally truncated modified collagenase having amino acid sequence as denoted by any one of SED ID NOs: 2-7; or any combination thereof. Each possibility is a separate embodiment.

In some embodiments, the recombinant collagenase comprises an amino acid sequence having at least about 65% sequence identity to an amino acid sequence of the N-terminally truncated reference collagenase having amino acid sequence as denoted by SED ID NO: 1 or SED ID NO: 9. Each possibility is a separate embodiment.

In some embodiments, the collagenase originates from Clostridium H (histolyticum) and/or Clostridium T (Tetani). Each possibility is a separate embodiment.

In some embodiments, the collagenase originates from Clostridium H (histolyticum).

In some embodiments, the recombinant collagenase polypeptide has an amino acid sequence as denoted by SEQ ID NO: 1 and/or SEQ ID NO: 9. Each possibility is a separate embodiment.

In some embodiments, the recombinant collagenase polypeptide comprises an amino acid sequence as denoted by any one of SED ID NO: 2, SED ID NO: 3, SED ID NO: 4, SED ID NO: 5, SED ID NO: 6, SED ID NO: 7, or any combination thereof. Each possibility is a separate embodiment.

In some embodiments, the recombinant collagenase polypeptide comprises an amino acid sequence as denoted by SED ID NO: 2 and/or SED ID NO: 5. Each possibility is a separate embodiment.

In some embodiments, the recombinant collagenase polypeptide comprises an amino acid sequence as denoted by SEQ ID NO: 8.

In some embodiments, the administration is local administration.

In some embodiments, the administration is by injection.

In some embodiments, the administration is to a connective tissue of marginal gingiva (lamina propria).

In some embodiments, the recombinant collagenase polypeptide or the composition comprising the same are configured to affect gingival fibers, thereby providing enzymatic fiberotomy. Each possibility is a separate embodiment.

In some embodiments, the recombinant collagenase polypeptide is administered at an effective amount of at least about 1.2 mg/ml. In some embodiments, the recombinant collagenase polypeptide is administered at an effective amount of at least about 4 mg/ml.

In some embodiments, the orthodontic procedure utilizing the recombinant collagenase polypeptide or the composition comprising the same, provides equal or enhanced effect on tooth movement (mm), compared to same orthodontic procedure utilizing surgical fiberotomy; and wherein the orthodontic procedure comprises accelerated orthodontic tooth movement (OTM). Each possibility is a separate embodiment.

In some embodiments, the orthodontic procedure utilizing the recombinant collagenase polypeptide or the composition comprising the same, provides equal or improved reduction in relapse compared to relapse after same orthodontic procedure utilizing surgical fiberotomy; and wherein the orthodontic procedure comprises prevention of post-orthodontic relapse. Each possibility is a separate embodiment.

According to some embodiments, the collagenase may be selected from a bacterial collagenase and mammalian Matrix Metallopeptidase (MMP). Each possibility is a separate embodiment. In some embodiments, the bacterial collagenase may be of G, H or T type.

According to some embodiments, the collagenase may be a non-naturally occurring, modified recombinant collagenase. In some embodiments, a modified recombinant collagenase exhibit increased solubility and/or thermal resistance and/or thermal stability, as compared to a WT collagenase protein.

In some embodiments, a modified recombinant collagenase may include one or more truncations and/or one or more amino acid substitutions, in particular surface exposed amino acids. In some embodiments, the one or more amino acid substitutions may include the substitution of hydrophobic (or at least less hydrophilic) amino acids to more hydrophilic or charged amino acids.

According to some embodiments, as exemplified herein, utilizing the methods and compositions disclosed herein, various orthodontic procedures may be facilitated in a safe, convenient, cost-effective manner, with reduced side effects, optionally replacing surgical procedures involved in such orthodontic procedures.

According to some embodiments, enzymatic degradation of collagen utilizing the compositions disclosed herein can be used for accelerated orthodontic tooth movement (OTM), teeth alignment and/or prevention of post-orthodontic treatment relapse (for example, by replacing the use of a physical surgical scalpel for the circumferential supracrestal fiberotomy), by being administered into the connective tissue of the marginal gingiva (lamina propria), in order to degrade the gingival fibers, as further detailed below.

According to some embodiments, advantageously, in contrast to a surgical fiberotomy currently used for orthodontic procedures, the enzymatic fiberotomy as disclosed herein, is performed in a minimally invasive manner, without surgical intervention that can compromise healthy tissues. Moreover, such enzymatic fiberotomy can expand the therapeutic spectrum for patients with coagulation problems and prevent complications such as discomfort and post-operative pain, which are associated with surgical procedures.

In some embodiments, a wild type (non-modified) collagenase polypeptide may have an amino acid sequence as denoted SEQ ID NO: 8.

In some embodiments, a recombinant collagenase polypeptide having a truncation of an N-terminal region thereof has an amino acid sequence as denoted by SEQ ID NO: 1.

In some embodiments, a modified recombinant collagenase polypeptide may have an amino acid sequence as denoted by any one of SEQ ID NO: 2, SEQ ID NO: 3, SEQ ID NO: 4, SEQ ID NO: 5, SEQ ID NO: 6 or SEQ ID NO: 7. Each possibility is a separate embodiment.

According to some embodiments, modified recombinant collagenase may be thermally stable as compared to a WT collagenase or to un-modified collagenase.

According to some embodiments, provided herein are methods and compositions for use in orthodontic treatments, the methods include administration of a collagenase (WT recombinant and/or modified recombinant collagenase (that may include a truncation and/or one or more point mutations), to a subject in need thereof. In some embodiments, the administration is performed to a connective tissue of the marginal gingiva.

According to some embodiments, a modified recombinant collagenase polypeptide, may include one or more amino acid replacements relative to the corresponding wild-type collagenase amino acid sequence and/or an N-terminal and/or a C-terminal truncation compared to the corresponding WT collagenase. Each possibility is a separate embodiment.

In some embodiments, the N-terminal and/or C-terminal truncation may include a truncation of at least 1, at least 2, at least 10, at least 15, at least 50, at least 100, at least 118, at least 119, at least 120, or more amino acids relative to the corresponding wild type collagenase amino acid sequence. Each possibility is a separate embodiment.

According to some embodiments, the modified recombinant collagenase polypeptide may further include a Tag sequence at the N-terminus and/or the C-terminus thereof. In some embodiments, a linker may be placed between the tag sequence and the collagenase polypeptide sequence.

According to some embodiments, the recombinant collagenase polypeptide (WT or modified) or the composition may be administered locally. In some embodiments, the administration may be by local injection. According to some embodiments, the administration may degrade gingival fibers (for example, when used for prevention of post treatment relapse). In some embodiments, the administration is not to the PDL region.

According to some embodiments, there is provided a method of orthodontic treatment in a subject in need thereof, the method may include administering to the subject in need thereof an effective amount of one or more: of: a wt collagenase, a recombinant collagenase polypeptide, a modified recombinant collagenase polypeptide, or a composition including one or more of the collagenases. According to some embodiments, the administration may be local administration, for example, into a connective tissue of the marginal gingiva.

According to some embodiments, there is provided a method of treating an orthodontic condition in a subject in need thereof, the method may include administering to the subject in need thereof a therapeutically effective amount of a recombinant collagenase polypeptide, or a composition including the same, wherein the recombinant collagenase polypeptide may include a truncation of an N-terminus thereof and optionally one or more amino acid substitutions or deletions, as compared to a corresponding WT collagenase.

According to some embodiments, there is provided a recombinant collagenase polypeptide, or a composition comprising the same, for use in orthodontic procedures in a subject in need thereof. In some embodiments, the orthodontic procedures is selected from accelerated orthodontic tooth movement (OTM), post-orthodontic relapse prevention and teeth alignment. Each possibility is separate embodiment.

In some embodiments, the Collagenase is selected from WT, truncated and modified collagenase. In some embodiments, the collagenase originates from Clostridium G, H, or T Collagenase. In some embodiments, the collagenase polypeptide has an amino acid sequence as denoted by SEQ ID NO: 1. In some embodiments, the collagenase polypeptide has an amino acid sequence as denoted by any one of SEQ ID NOs: 2-7. In some embodiments, the collagenase polypeptide has an amino acid sequence as denoted by SEQ ID NO: 8.

In some embodiments, the recombinant collagenase polypeptide or the composition are administered locally. In some embodiments, administration is by local injection. In some embodiments, the administration is localized to a connective tissue of marginal gingiva (lamina propria). In some embodiments, the recombinant collagenase polypeptide or the composition comprising the same are configured to affect gingival fibers.

According to some embodiments, there is provided a method of performing an orthodontic procedure in a subject in need thereof, the method comprising administering to the subject in need thereof an effective amount of a recombinant collagenase polypeptide, or a composition comprising the same.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1B show pictograms presenting a rat model for bilateral orthodontic tooth movement (OTM) wherein appliance was inserted and bonded into the first molars of rats in a split mouth design where one side is subjected to fiberotomy and the other acted as a control, according to some embodiments. Briefly, FIG. 1A shows a picture of a mouth of a representative rat that was subjected to the split mouth design, where randomly assigned one side of the rat mouth was subjected to a traditional surgical fiberotomy, or as disclosed herein an enzymatic fiberotomy (i.e., by using collagenase), and the other side received no treatment or phosphate-buffered saline (PBS) (control). FIG. 1B shows an intraoral view. Tooth movement is assessed before and after orthodontic tooth movement (OTM) procedure is performed in a split mouth design, as well as after relapse. The measurements of deviations were performed by superimposing intraoral digital scans as demonstrated in FIGS. 3A-3D.

FIG. 2 shows an illustration of administration location of a collagenase (or compositions comprising the same) when used for orthodontic procedures, according to some embodiments; Right side of the illustration: shows the herein disclosed local administration by injection of recombinant collagenase polypeptide, or injection of PBS, to a connective tissue of marginal gingiva (lamina propria) herein used for orthodontic procedures such as accelerated orthodontic tooth movement (OTM), post-orthodontic relapse prevention and teeth alignment. Left side of the illustration: shows state of the art approach for atraumatic tooth extraction utilizing local injection into the periodontal ligament (PDL).

FIGS. 3A-3D show illustrative images of intraoral digital scans used for the analysis of orthodontic tooth movement OTM and post-orthodontic relapse at days 35 and/or 42. Measurements of deviations in tooth movement were performed by superimposing digital scans of the upper intraoral cavity (maxilla) which were acquired before and after the OTM procedure (days 0 and 28) as well as after relapse (days 35 and 42), according to some embodiments. The scans were superimposed based on a reference structure (the palate) using computer software that then calculates the deviations. deviations in distances are measured between the palatal suture and the first molars (M1) contour.

FIG. 3A shows the scans superimposed based on the palatal surface. Shown are Horizontal view (I); Coronal view (II).

FIG. 3B shows segmentation of the scan into areas of interest. The palate (purple) and the left (red) and right (green) first molars.

FIG. 3C the software calculated the deviations between the scans and divided them into Under (Blue) and Over (Orange) Whisker vectors that demonstrate the movement. Shown are postoperative scan (I); preoperative scan (II); and aligned scan (III) showing the tilting and translation movement of the molar.

FIG. 3D. The software averaged all deviations and presented them on a color map. Shown is a control measure performed as a test to check for a deviation within the palate as a reference structure.

FIGS. 4A-4E show bar graphs presenting the degree of tooth movement in rats that were subjected to orthodontic procedure in a split-mouth design including appliance that was designed to apply equal orthodontic movement on both sides of the upper jaw utilizing a nickel-titanium (NiTi) wire. The split-mouth design included on the first molar of the treated side PBS injection while the contralateral side served as no treatment control (FIG. 4A); traditional surgical fiberotomy while the contralateral side served as no treatment control (FIG. 4B); enzymatic fiberotomy using recombinant N-terminally truncated collagenase (ColG) having the amino acid sequence denoted by SEQ ID NO: 1 performed on the first molar on one side while the contralateral side received either no treatment serving as control (FIG. 4C) or a traditional surgical fiberotomy (FIG. 4D; and enzymatic fiberotomy using commercial collagenase (C.COL) while the contralateral side served as no treatment control (FIG. 4E). Orthodontic force was applied for 28 days before measuring tooth movement, presented here as the mean orthodontic tooth movement (OTM) from T0 in mm. *p<0.05, **p<0.01.

FIGS. 5A-5E show bar graphs presenting the degree of post-orthodontic relapse in the same rats that were subjected to the orthodontic procedure in a split-mouth design and tooth movement measurements described in FIG. 4A-4E wherein the split-mouth design included on the first molar of the treated side PBS injection while the contralateral side served as no treatment control (FIG. 5A); traditional surgical fiberotomy while the contralateral side served as no treatment control (FIG. 5B); enzymatic fiberotomy using recombinant N-terminally truncated collagenase (ColG) having the amino acid sequence denoted by SEQ ID NO: 1 while the contralateral side received either no treatment serving as control (FIG. 5C) or a traditional surgical fiberotomy (FIG. 5D); and enzymatic fiberotomy using commercial collagenase (C.COL) while the contralateral side served as no treatment control (FIG. 5E). Orthodontic force was applied for 28 days followed by 14 days (up to day 42) without force before measuring relapse, presented here as ratio from deactivation. *p<0.05, **p<0.01.

FIG. 6 shows lines graphs of thermal stability of recombinant N-terminally truncated modified collagenase (Des1) having the amino acid sequence denoted by SEQ ID NO: 5 compared to the recombinant N-terminally truncated collagenase (ColG) (SEQ ID NO: 1). A heat inactivation assay was performed by preincubating the purified recombinant proteins, at temperatures ranging between 35 and 90° C. for 1 h. Residual activity was then measured by monitoring collagenase activity. The midpoint of temperature inactivation is the temperature at which 50% of the activity was retained (T50).

FIG. 7 shows a bar graph presenting collagenase activity in-vitro. A single concentration of collagen was incubated with the recombinant N-terminally truncated collagenase (ColG) (SEQ ID NO: 1) or the commercial collagenase C.COL. Assay specificity was validated via the depletion of the different reaction components including depletion of substrate (w/o collagen), or depletion of DHPAA-3,4-dihydroxyphenylacetic acid that interacts with peptides with N-terminus Gly after collagen degradation (w/o DHPAA), or depletion of enzyme (w/o collagenase) from the reaction. The two right bars (ColG and C.COL) show the full reaction in the presence of all the components required for the reaction. The mean and standard deviation (SD) of the in vitro assays were statistically analyzed using a one-way ANOVA with Tukey multiple correction. (*p<0.05, ****p<0.0001).

FIG. 8 illustrate the NITI closed coil that is glued to the incisor and the occlusal surface of the first molar of rats.

### DETAILED DESCRIPTION OF THE INVENTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

Unless otherwise defined, all technical and scientific terms used herein have the same meaning as commonly understood by one of ordinary skill in the art to which this disclosure pertains.

As used herein, the singular forms “a”, “an” and “the” are intended to include the plural forms as well, unless the context clearly indicates otherwise. “a” and “an” are used herein to refer to one or more than one (i.e., to at least one) of the stated object, unless the context clearly dictates otherwise. By way of example, “a collagenase” means one or more collagenase(s).

As used herein, the term “about” when referring to a measurable value such as an amount, a temporal duration, and the like, is meant to encompass deviations/variations of +20% or in some embodiments ±10%, or in some embodiments ±5%, or in some embodiments ±1%, or in some embodiments ±0.1% from the specified value, as such deviations are appropriate to perform the disclosed methods.

As used herein, the term “comprising” is synonymous with the terms “including,” “containing,” or “characterized by,” and is inclusive or open-ended i.e. does not exclude additional, unrecited elements. According to some embodiments, the term comprising may be replaced with the term with the term “consisting of” which excludes any element, step, or ingredient not specified in the claim. According to some embodiments, the term comprising may be replaced with the term “consisting essentially of” which limits the scope of a claim to the specified materials or steps “and those that do not materially affect the basic and novel characteristics” of the claimed invention.

As used herein, the terms “prevent”, “reduce”, “attenuate”, “ameliorate”, “alleviate”, and “inhibit” are used interchangeably.

As used herein, the terms “enhanced”, “increased”, “elevated” are used interchangeably.

As used herein, the terms “subject”, “patient” or “individual” may be used interchangeably and generally refer to a human, although the methods of the invention are not necessarily limited to humans and should be useful in other mammals or non-mammal animals including for example, but not limited to farm animals, pets, and the like.

According to some embodiments, “a subject in need thereof” includes a subject undergoing an orthodontic procedure including, for example, but not necessarily limited to orthodontic tooth movement (OTM), prevention of post-orthodontic relapse and tooth alignment.

As used herein, the term “treating” refers to an approach for obtaining beneficial or desired results, including clinical results in treating a subject undergoing an orthodontic procedure. Beneficial or desired clinical results can include, but are not limited to, prevention, alleviation, amelioration or reduction of one or more symptoms or conditions, diminishment of the extent of the disease or condition, stabilization of the state of the disease or condition, prevention of deterioration of the disease or condition, delay or slowing of disease/condition progression, amelioration or palliation of the disease state, and remission (whether partial or total).

The term “treatment” as used herein refers to both therapeutic treatment and prophylactic or preventative measures, including for example medical intervention in the form of pharmaceuticals/compositions or surgery. In some embodiments, those in need of treatment include those already having a condition as well as those in which the condition is to be prevented.

As used herein, the term “administering” includes routes of administration which allow the compositions of the invention to perform their intended function. A variety of routes of administration are possible including, but not necessarily limited to, local administration, preferably by injection, even more preferably local injection to the marginal gingiva (lamina propria).

The terms “polypeptide,” “peptide” and “protein” are used interchangeably herein to refer to a polymer of amino acid residues. The terms also apply to amino acid polymers in which one or more amino acid residue is an artificial chemical analogue of a corresponding naturally occurring amino acid, as well as to naturally occurring amino acid polymers. In some embodiments, one or more of amino acid residue in the polypeptide may contain modification, such as but be not limited only to, glycosylation, phosphorylation or disulfide bond shape. Also provided are conservative amino acid variants and homologs of the peptides and protein molecules disclosed herein. Variants according to the invention also may be made that conserve the overall molecular structure of the encoded proteins or peptides, and may include non-conservative as well as conservative substitutions. In addition, the invention encompasses natural homologs that may conserve the overall molecular structure of the encoded proteins or peptides, and may include non-conservative as well as conservative substitutions. Given the properties of the individual amino acids comprising the disclosed protein products, some rational substitutions will be recognized by the skilled worker. “conservative substitutions” may be made, for instance, on the basis of similarity in polarity, charge, solubility, hydrophobicity, hydrophilicity, and/or the amphipathic nature of the residues involved. As used herein, amino acids and peptide sequences are marked using conventional amino acid nomenclature (single letter or 3-letters code). For example, amino acid “Serine” may be marked as “Ser” or “S” and amino acid “Cysteine” may be marked as “Cys” or “C”.

As used herein, the term “conservative amino acid substitutions” is related to the term “sequence similarity” and “sequence homology” and refers to recombinant collagenases having one or more of their amino acid residues changed into another amino acid exhibiting similar or equivalent biochemical, structural and/or chemical properties therefore the change is considered as a conservative replacement that preserve the overall molecular structure of the encoded proteins or peptides. “conservative substitutions” or “conservative replacement” may be used interchangeably with the term “conservative amino acid substitutions”.

For example, amino acids may be grouped into six main classes on the basis of their structure and the general chemical characteristics of their side chains (R groups).
Non-polar or Hydrophobic: Glycine (G), Alanine (A), Valine (V), Isoleucine (I), Leucine (L), Methionine (M).
Non-polar or hydrophobic/Aromatic: Phenylalanine (F), Tyrosine (Y), Tryptophan (V).
Polar uncharged: Serine(S), Threonine (T), Asparagine (N), Glutamine (Q), Cysteine (C) or Selenocysteine, Proline (P)
Basic/Positively charged: Histidine (H), Lysine (K), Arginine (R)
Acidic/Negatively charged: Aspartate (D), Glutamate (E).

Each of the above-mentioned classes contains amino acids that are conservative substitutions for one another due to similar biochemical, structural and/or chemical properties. Based on this generally accepted grouping, conservative replacements may include, for example, but are not necessarily limited to:
Based on structure/size or non-polarity: Alanine (A) and Glycine (G) may be considered as conservative replacement; Based on structure/size or non-polarity: Valine (V), Isoleucine (I), Leucine (L), Methionine (M) may be considered as conservative replacement; Based on structure/size or non-polarity/hydrophobicity: Phenylalanine (F), Tyrosine (Y), Tryptophan (W), and possibly also Histidine (H) may be considered as conservative replacement; Based on charge: Aspartate (D) and Glutamate (E) may be considered as conservative replacement; Based on charge: Lysine (K) and Arginine (R) may be considered as conservative replacement; Based on structure/size or polarity: Asparagine (N) and Glutamine (Q) may be considered as conservative replacement; Based on structure/size or polarity: Serine(S) and Threonine (T) may be considered as conservative replacement; Based on containing sulfur Cysteine (C) and Methionine (M) may be considered as conservative replacement;

As used herein the terms “sequence homology” or “sequence similarity” or “sequence identity” refer to the resemblance i.e., the level/percentage of similarity or the level/percentage of identity between two or more collagenase protein sequences (at least one of them being the “reference collagenase” or “corresponding collagenase”) when aligned together and compared using a common sequence alignment tool or multiple sequence alignment tool (MSA) for optimal matching of amino acid residues (for example, alignment of collagenase protein from different origins (mammalian, non-mammalian, human, or Clostridium origin; or for example of full-length vs. truncated forms of same collagenase).

The percentage % of sequence identity refers to the amount of amino acid residues which match exactly (same amino acid) between two different sequences optimally aligned over a comparison window that may comprise additions or deletions (i.e., gaps) as compared to the reference sequence (which does not comprise additions or deletions). Hereby, gaps are not counted, and the measurement is relational to the shorter of the two sequences.

The percentage % of sequence similarity refers to the amount of amino acid residues which match exactly (same amino acid) and to the amount of amino acid residues which are conserved substitutions having similar physicochemical properties, between two different sequences optimally aligned over a comparison window that may comprise additions or deletions (i.e., gaps) as compared to the reference sequence (which does not comprise additions or deletions). Hereby, gaps are not counted, and the measurement is relational to the shorter of the two sequences.

However, it is recognized that homologous protein will often exhibit aligned positions where the amino acid are not identical but are conservative amino acid substitutions, where the amino acid is replaced for a different amino acid having similar or equivalent biochemical, structural and/or chemical properties and therefore do not change the structure and/or functional of the protein. In such a case, for example, a certain percentage of sequence identity may translate into a higher percentage of sequence similarity.

The present disclosure provides at least three different types of recombinant collagenases including WT collagenase, truncated collagenase, and truncated modified collagenase, or compositions comprising the same. The recombinant forms of the collagenases may include an artificial N-terminus region introducing a tag/an affinity tag and/or a protease cleavage site to enable identification and purification.

In some embodiments, the recombinant collagenase polypeptide comprises one or more collagenases selected from: a full-length WT collagenase, N-terminally truncated un-modified collagenase, and N-terminally truncated modified collagenase, or any combination thereof.

In some embodiments, the orthodontic procedures comprise one or more procedures selected from accelerated orthodontic tooth movement (OTM), post-orthodontic relapse prevention and teeth alignment, or any combination thereof.

In some embodiments, the orthodontic procedure utilizing the recombinant collagenase polypeptide or the composition comprising the same, provides equal or enhanced effect on tooth movement (mm), compared to same orthodontic procedure utilizing surgical fiberotomy; and wherein the orthodontic procedure comprises accelerated orthodontic tooth movement (OTM).

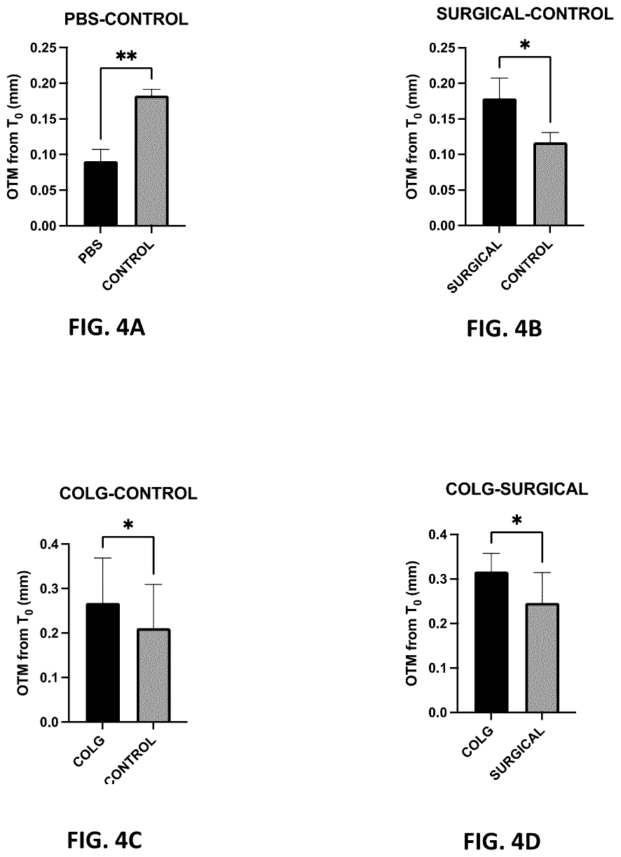

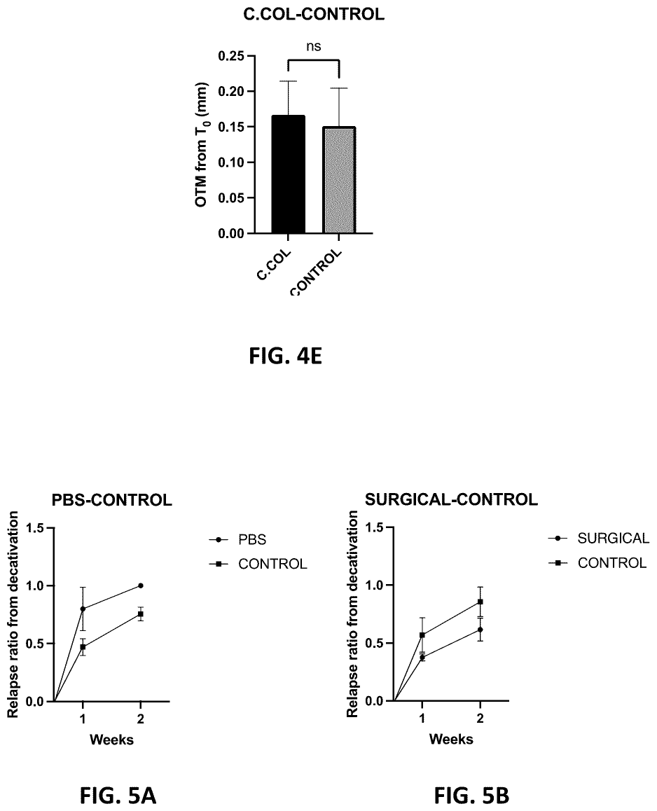

For example, when comparing enzymatic fiberotomy using COLG to surgical fiberotomy, COLG showed at least a similar potential, and possibly even an enhanced ability for acceleration of tooth movement. In some embodiments, when directly comparing enzymatic fiberotomy using COLG to surgical fiberotomy, COLG showed enhanced orthodontic movement by a mean of 32.3±18.3%. reference is made to FIG. 4D.

In some embodiments, the orthodontic procedure utilizing the recombinant collagenase polypeptide or the composition comprising the same, provides equal or improved reduction in relapse compared to relapse after same orthodontic procedure utilizing surgical fiberotomy; and wherein the orthodontic procedure comprises prevention of post-orthodontic relapse.

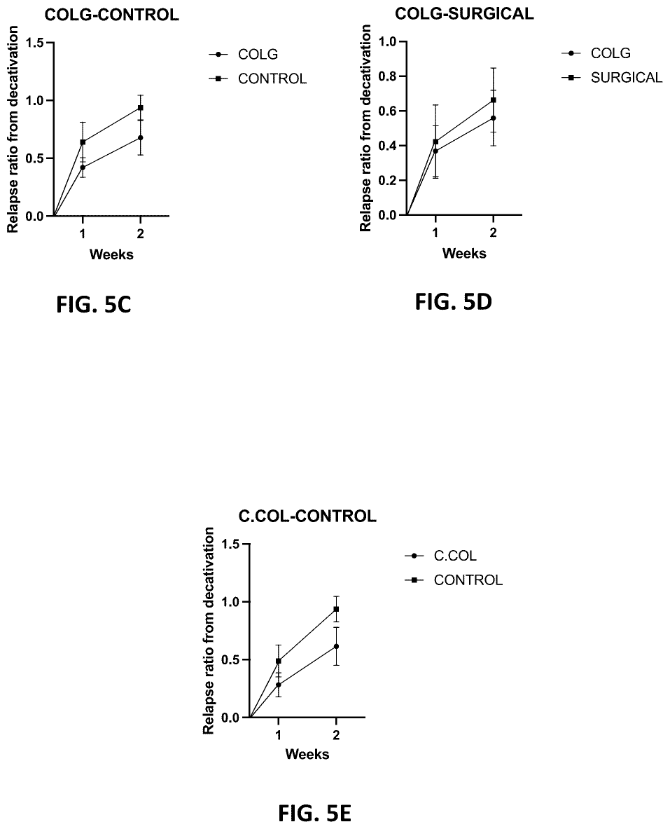

Reference is now made to FIGS. 4A-4E; Example 1 and FIGS. 5A-5E; Example 2, exemplifying the use of recombinant collagenase for orthodontic procedures and demonstrating the effect of the recombinant collagenase on orthodontic tooth movement (OTM) and prevention of post-orthodontic relapse.

In some embodiments, the WT collagenase, truncated collagenase, and truncated modified collagenase are from a non-mammalian origin. Each possibility is a separate embodiment.

In some embodiments, the WT collagenase, truncated collagenase, and truncated modified collagenase are from mammalian origin. Each possibility is a separate embodiment.

In some embodiments, the WT collagenase, truncated collagenase, and truncated modified collagenase are of human origin. Each possibility is a separate embodiment.

In some embodiments, the WT collagenase, truncated collagenase, and truncated modified collagenase are of Clostridium origin (Clostridium sp.). Each possibility is a separate embodiment.

In some embodiments, Clostridium sp. include but are not limited to Clostridium H (histolyticum) and Clostridium T (Tetani).

In some embodiments, the WT collagenase, truncated collagenase, and truncated modified collagenase are of Clostridium origin (Clostridium sp.) selected from Clostridium H (histolyticum) and Clostridium T (Tetani) Collagenase. Reference is made to Example 6.

In some embodiments, the WT collagenase, truncated collagenase, and truncated modified collagenase are of Clostridium. H origin (Clostridium histolyticum). Each possibility is a separate embodiment.

The terms “wild type (WT) collagenase”, “full-length WT collagenase”, ““WT un-modified collagenase”, and “recombinant WT collagenase” may interchangeably be used. The terms may refer to a full-length recombinant collagenase protein from Clostridium histolyticum comprising an amino acid sequence as denoted by SEQ ID NO: 8 (corresponding to Accession No. Q9X721), or in some embodiments, the terms may refer to homologous collagenase (for example but not necessarily of mammalian, non-mammalian, human, or Clostridium origin) comprising at least 65% up to 100% sequence identity to that of the reference WT collagenase having the amino acid sequence as denoted by SEQ ID NO: 8 (Accession No. Q9X721). Each possibility is a separate embodiment.

As used herein the terms, “N-terminally truncated recombinant collagenase”, “truncated recombinant collagenase”, “N-terminally truncated collagenase”, “un-modified truncated collagenase”, and “ColG” may interchangeably be used. The terms relate to a WT Collagenase having a truncation of the first 118 amino acids (e.g., the ColG, has an N-terminus truncation), or in some specific instances a truncation of less than the first 118 amino acid of the N-terminus. The terms may refer to a truncated collagenase protein comprising an amino acid sequence as denoted by SEQ ID NO: 1 (or SEQ ID NO: 9), or in some embodiments, the terms may refer to homologous collagenase (for example but not necessarily of mammalian, non-mammalian, human, or Clostridium origin) comprising at least 65% and up to 100% sequence identity to that of the reference truncated collagenase protein having the amino acid sequence as denoted by SEQ ID NO: 1 or SEQ ID NO: 9. Each possibility is a separate embodiment.

In some embodiments, the N-terminal truncation includes truncation of 118 amino acids or less. Each possibility is a separate embodiment. In some embodiments, the N-terminal truncation includes truncation of between at least 1 amino acid and 125 amino acids. In some embodiments, the N-terminal truncation includes truncation of between at least 1 amino acid and 118 amino acids.

In some embodiments, the N-terminal truncation includes truncation of less than 119 amino acids, less than 115 amino acids, less than about 100 amino acids, less than about 50 amino acids, less than about 25 amino acids, less than 10 amino acids, less than 5 amino acids, or less than 2 amino acids. In some embodiments, the N-terminal truncation includes truncation of between about the first amino acid and about the first 118 amino acids relative to the reference or corresponding wild type collagenase amino acid sequence. In some embodiments, the N-terminal truncation includes truncation of between about the first 20 amino acids and about the first 118 amino acids. Each possibility is a separate embodiment.

In some embodiments, the N-terminal and/or C-terminal truncation may include a truncation of at least 1 amino acid, at least 2 amino acids, at least 10 amino acids, at least 15 amino acids, at least 50 amino acids, at least 100 amino acids, at least 118 amino acids, at least 119 amino acids, at least 120 amino acids, at least 125 amino acids, or more amino acids relative to the reference or corresponding wild type collagenase amino acid sequence. Each possibility is a separate embodiment.

In some embodiments, the N-terminal truncation comprises truncation of at least 118 amino acid residues, with respect to the corresponding full-length WT collagenase. Each possibility is a separate embodiment.

As used herein the terms “modified collagenase”, “modified recombinant collagenase”, “mutated recombinant collagenase”, “N-terminally truncated modified collagenase”, and “Des” may interchangeably be used. The terms relate to a truncated collagenase that was further modified by introduction of point mutations/amino acid substitutions. The terms may also relate to naturally modified (e.g., homolog) or artificially modified (e.g., variant) forms of a truncated recombinant collagenase such as the ColG having an N-terminus truncation). The terms may refer to a recombinant truncated modified collagenase protein comprising an amino acid sequence as denoted by SEQ ID NOs: 2-4 (or by SEQ ID NO: 5-7), or in some embodiments, the terms may refer to homologous collagenase (for example but not necessarily of mammalian, non-mammalian, human, or Clostridium origin) comprising at least 65% and up to 100% sequence identity to that of the corresponding truncated modified collagenase protein having the amino acid sequence as denoted by any one of SEQ ID NOs: 2-7 or to the reference truncated collagenase protein having the amino acid sequence as denoted by SEQ ID NO: 1 or SEQ ID NO: 9. Each possibility is a separate embodiment.

In some embodiments, the modified recombinant collagenase differs from the corresponding wild-type collagenase or the un-modified truncated recombinant collagenase by at least one mutation selected from amino acid substitution(s) (i.e., point mutations) and/or deletions(s). In some embodiments, the modifications/mutations may increase its thermo-stability with respect to the un-modified truncated collagenase or the WT collagenase. In some embodiments, the truncated modified recombinant collagenase differs from the corresponding truncated un-modified collagenase by at least one mutation selected from amino acid substitution(s), and/or deletions(s).

In some embodiments, the modified recombinant collagenase includes an amino acid sequence as denoted by SEQ ID NO: 2 (also referred to herein as Des1). In some embodiments, the modified recombinant includes an amino acid sequence as denoted by SEQ ID NO: 3 (also referred to herein as Des4). In some embodiments, the modified recombinant collagenase includes an amino acid sequence as denoted by SEQ ID NO: 4 (also referred to herein as Des6). In some embodiments, the modified recombinant collagenase includes an amino acid sequence as denoted by SEQ ID NO: 5 (Des1). In some embodiments, the modified recombinant collagenase includes an amino acid sequence as denoted by SEQ ID NO: 6 (Des4). In some embodiments, the modified recombinant collagenase includes an amino acid sequence as denoted by SEQ ID NO: 7 (Des6). In some embodiments, a modified recombinant collagenase of an origin other than Clostridium may include a corresponding point mutation and/or deletion in the respective WT collagenase, which are equivalent or homologous to the mutations introduced in the Clostridium WT collagenase.

In some embodiments, the modified recombinant collagenase differs from the wild-type collagenase or the un-modified truncated recombinant collagenase by at least one mutation selected from the mutations of the herein disclosed Table 2, in Example 3; and wherein the at least one mutation increases its thermo-stability with respect to the un-modified truncated collagenase or the WT collagenase.

In some embodiments, the truncated modified recombinant collagenase differs from the corresponding wild-type collagenase or from the corresponding truncated un-modified recombinant collagenase by at least one amino acids substitution or deletion selected from a mutation(s) listed in Table 2; and wherein the at least one mutation enhances its thermo-stability with respect to the corresponding N-terminally truncated un-modified collagenase or the corresponding full-length WT collagenase by at least 1.0° C.; and wherein the thermo-stability is calculated as a midpoint of temperature inactivation (T₅₀). Each possibility is a separate embodiment.

In some embodiments, the truncated modified recombinant collagenase differs from the wild-type collagenase or from the truncated un-modified recombinant collagenase by at least one amino acids substitution or deletion selected from a mutation(s) listed in Table 2; and wherein the at least one mutation enhances its thermo-stability with respect to the N-terminally truncated un-modified collagenase or the full-length WT collagenase by at least 1.0° C.; and wherein the thermo-stability is calculated as a midpoint of temperature inactivation (T50). Each possibility is a separate embodiment.

In some embodiments, the truncated modified recombinant collagenase differs from the wild-type collagenase or from the truncated un-modified recombinant collagenase by at least ₁ amino acids substitution, at least 2 amino acids substitution, at least 3 amino acids substitution, at least 4 amino acids substitution, at least 5 amino acids substitution, at least 6 amino acids substitution, at least 7 amino acids substitution, at least 8 amino acids substitution, at least 9 amino acids substitution, at least 10 amino acids substitution, at least 11 amino acids substitution, at least 12 amino acids substitution, at least 13 amino acids substitution, at least 14 amino acids substitution, or 15 amino acids substitution, selected from a mutation(s) listed in Table 2. Each possibility is a separate embodiment.

In some embodiments, the truncated modified recombinant collagenase differs from the wild-type collagenase or from the truncated un-modified recombinant collagenase by between 1 and 15 mutations.

In some embodiments, the at least 1 amino acids substitution, at least 2 amino acids substitution, at least 3 amino acids substitution, at least 4 amino acids substitution, at least 5 amino acids substitution, at least 6 amino acids substitution, at least 7 amino acids substitution, at least 8 amino acids substitution, at least 9 amino acids substitution, at least 10 amino acids substitution, at least 11 amino acids substitution, at least 12 amino acids substitution, at least 13 amino acids substitution, at least 14 amino acids substitution, or 15 amino acids substitution, selected from a mutation(s) listed in Table 2, enhances its thermo-stability with respect to the N-terminally truncated un-modified collagenase or the full-length WT collagenase by at least 1.0° C.; and wherein the thermo-stability is calculated as a midpoint of temperature inactivation (Tso). Each possibility is a separate embodiment.

In some embodiments, the mutations are selected from substitution and or deletion of an amino acid at position A334, A458, A709, D536, D737, E710, F295, G670, G672, M183, N149, N203, N287, Q669, S353, S701, T635, or any combination thereof; wherein the positions are with respect to the WT collagenase having amino acid sequence as denoted by SEQ ID NO: 8. Each possibility is a separate embodiment.

In some embodiments, the mutations are selected from A334D, A458P, A709E, D536P, D737K, E710H, F295Y, G670N, G672T, M183D, N149Q, N203Y, N287Y, Q669D, S353H,

S701N, T635N, or any combination thereof; wherein the positions are with respect to the WT collagenase having amino acid sequence as denoted by SEQ ID NO: 8. Each possibility is a separate embodiment.

In some embodiments, the mutations are selected from F295Y, A334D, S353H, T635N, Q669D, G670N, G672T, S701N and A709E; or any combination thereof; wherein the positions are with respect to the WT collagenase having amino acid sequence as denoted by SEQ ID NO: 8. Each possibility is a separate embodiment.

In some embodiments, the at least one mutation enhances its thermo-stability with respect to the N-terminally truncated un-modified collagenase or the full-length WT collagenase by at least about 1.0° C., at least about 1.2° C., at least about 1.5° C., at least about 2.0° C., or at least about 2.5° C., and wherein the thermo-stability is calculated as a midpoint of temperature inactivation (T50). Each possibility is a separate embodiment.

In some embodiments, the thermo-stability of the N-terminally truncated modified collagenase is characterized by a midpoint of temperature inactivation (T₅₀) of at least about 54° C., at least about 54.5° C., at least about 55° C., at least about 55.5° C., or at least about 56° C., or at least about 56° C. Each possibility is a separate embodiment.

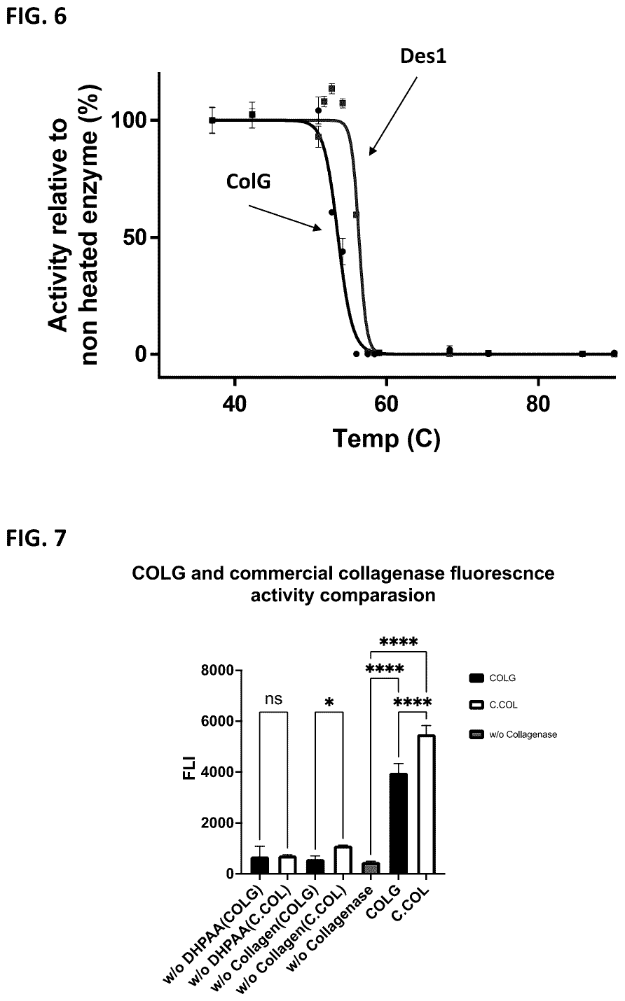

Reference is made to Table 2; Example 3 and FIG. 6; Example 4 describing the recombinant modified collagenases and their increased thermo-stability.

In some embodiments, the recombinant collagenase polypeptide comprises an amino acid sequence having at least about 65% sequence identity to an amino acid sequence of a corresponding collagenase; wherein the corresponding collagenase comprises one or more collagenases selected from: a full-length WT collagenase having amino acid sequence as denoted by SED ID NO: 8, an N-terminally truncated collagenase having amino acid sequence as denoted by SED ID NO: 1 or SED ID NO: 9, and N-terminally truncated modified collagenase having amino acid sequence as denoted by any one of SED ID NOs: 2-7; or any combination thereof. Each possibility is a separate embodiment.

In some embodiments, the recombinant collagenase polypeptide comprises an amino acid sequence having at least about 65% sequence identity to an amino acid sequence of the reference collagenase having amino acid sequence as denoted by any one of SED ID NO: 1, SED ID NO: 8, or SED ID NO: 9, or any combination thereof. Each possibility is a separate embodiment.

In some embodiments, the recombinant collagenase polypeptide comprises an amino acid sequence having at least about 65% sequence identity to an amino acid sequence of the N-terminally truncated reference collagenase having amino acid sequence as denoted by SED ID NO: 1 and/or SED ID NO: 9.

In some embodiments, the recombinant collagenase polypeptide comprises an amino acid sequence having at least about 65% sequence identity to an amino acid sequence of the full-length reference collagenase having amino acid sequence as denoted by SED ID NO: 8.

In some embodiments, the recombinant collagenase protein has at least about 65%, at least about 70%, at least about 75%, at least about 80%, preferably at least about 85%, at least about 86%, at least about 87%, at least about 88%, at least about 89%, at least about 90%, at least about 91%, at least about 92%, at least about 93%, at least about 94%, at least 95%, at least about 96%, at least about 97%, at least about 98%, at least 99%, at least 99.9%, or 100% sequence identity to the corresponding collagenase. Each possibility is a separate embodiment.

In some embodiments, the recombinant collagenase protein has at least about 65%, at least about 70%, at least about 75%, at least about 80%, preferably at least about 85%, at least about 86%, at least about 87%, at least about 88%, at least about 89%, at least about 90%, at least about 91%, at least about 92%, at least about 93%, at least about 94%, at least 95%, at least about 96%, at least about 97%, at least about 98%, at least 99%, at least 99.9%, or 100% sequence identity to an amino acid sequence of the N-terminally truncated reference collagenase having amino acid sequence as denoted by SED ID NO: 1 and/or SED ID NO: 9. Each possibility is a separate embodiment.

In some embodiments, the recombinant collagenase protein has between about 65% and 100% sequence identity to the corresponding collagenase. In some embodiments, the recombinant collagenase protein has between about 65% and 97.8% sequence identity to the corresponding collagenase. In some embodiments, the recombinant collagenase protein has between about 75% and 97.8% sequence identity to the corresponding collagenase. In some embodiments, the recombinant collagenase protein has between about 85% and 97.8% sequence identity to the corresponding collagenase. In some embodiments, the recombinant collagenase protein has between about 90% and 97.8% sequence identity to the corresponding collagenase. In some embodiments, the recombinant collagenase protein has between about 95% and 97.8% sequence identity to the corresponding collagenase.

In some preferred embodiments, the reference collagenase has the amino acid sequence denoted by SEQ ID NO: 1, SEQ ID NO: 8, or SEQ ID NO: 9, or any combination thereof; In even more preferred embodiments, the reference collagenase has the amino acid sequence denoted by SEQ ID NO: 1, or SEQ ID NO: 9, or any combination thereof. Each possibility is a separate embodiment.

In some embodiments, the recombinant collagenases are protein homologs of the reference collagenase.

The term “homologous proteins” has the meanings normally ascribed to it in the art.

In some embodiments, the homologous collagenase proteins of mammalian, non-mammalian, human, or Clostridium origin have at least about 65% sequence identity, at least about 75% sequence identity, at least about 80% sequence identity, at least about 85% sequence identity, at least about 90% sequence identity, at least 95% sequence identity, at least 96% sequence identity, at least 97% sequence identity, at least about 98% sequence identity, at least 99% sequence identity, at least 99.9% sequence identity, or 100% sequence identity, to the corresponding collagenase such as the WT reference collagenase (SEQ ID NO: 8) or the truncated reference collagenase (SEQ ID NO: 9 or SEQ ID NO: 1) or the mutated reference collagenase

(SEQ ID NOs: 2-7) originated from Clostridium histolyticum, or to any combination thereof. Each possibility is a separate embodiment.

The term “reference collagenase” may refer to a collagenase originated from Clostridium histolyticum, which in some embodiments, has the amino acid sequence denoted by any one of SEQ ID NO: 1-9, or any combination thereof.

The terms “reference collagenase” or “corresponding collagenase” may be used interchangeably. Nevertheless, in some specific embodiments, the term “corresponding collagenase” may be used more broadly than the term “reference collagenase” to refer to collagenase originated from a corresponding species which in some embodiments may be any species, including mammalian, non-mammalian, human, or Clostridium origin. For example, in some embodiments wherein the N-terminal truncation comprises truncation of the first 118 amino acids, or less, with respect to the corresponding full-length WT collagenase—the truncated collagenase includes collagenase originated from any species, including mammalian, non-mammalian, human, or Clostridium origin and the full-length WT collagenase is originated from the same species corresponding to the species of the truncated collagenase. In another example, wherein the truncated modified recombinant collagenase differs from the corresponding wild-type collagenase or from the corresponding truncated un-modified recombinant collagenase by at least one amino acids substitution or deletion selected from a mutation(s) listed in Table 2—the truncated modified recombinant collagenase includes collagenase originated from any species, including mammalian, non-mammalian, human, or Clostridium origin and the full-length WT collagenase or the truncated un-modified recombinant collagenase is originated from the same species corresponding to the species of the truncated collagenase.

In some embodiments, the corresponding collagenase comprises the reference collagenase.

The term “recombinant protein” as used herein have the meanings normally ascribed to it in the art. Artificial protein tag/an affinity tag and/or a protease cleavage site are commonly introduced to the C-terminal or the N-terminal regions of the protein to enable identification and purification of the recombinant form of the protein.

In preferred embodiments, the recombinant protein may be encoded by recombinant DNA technology. In less preferred embodiments, the collagenases protein may include chemically synthesized collagenases.

In some embodiments, the recombinant WT collagenase, recombinant truncated collagenase, and recombinant truncated modified collagenase comprise isolated or purified collagenase.

As used herein, the term “isolated” means either: 1) separated from at least some of the components with which it is usually associated in nature with respect of the Wild-Type collagenase; 2) prepared or purified by a process that involves the hand of man; 3) not occurring in nature.

According to some embodiments, the recombinant WT, truncated un-modified or the modified recombinant collagenases may further include a protein tag. As used herein, the term “tag” or “protein tag” refers to a peptide sequence bound to the N-terminus or C-terminus of the protein. According to some embodiments, the protein tag may include a glycoprotein. According to some embodiments, the protein tag may be used for separation, purification and/or identification/tracking of the tagged protein. Non-limiting examples of protein tags include: Myc-Tag, Human influenza hemagglutinin (HA), Flag-Tag, His-Tag, Glutathione-S-Transferase (GST) and a combination thereof. Each possibility represents a separate embodiment of the present invention. In some embodiments, the protein tag is His-tag.

According to some embodiments, the recombinant WT, truncated un-modified and modified recombinant collagenase may include a protein tag upon production, which may be consequently cleaved and/or removed from the produced recombinant collagenase prior to incorporation into a composition or prior to being introduced to cells/administered. Each possibility is a separate embodiment. Cleavage and/or removal of a tag may be performed by any method known in the art, such as, but not limited to, enzymatic and/or chemical cleaving. Each possibility is a separate embodiment. In some embodiments, the cleavage may be facilitated by a cleavage site included the amino acid sequence. In some embodiments, the truncated un-modified recombinant and/or the modified recombinant collagenases include at the N-terminus a tag sequence and a cleavage site. Each possibility is a separate embodiment.

According to some embodiments, the recombinant WT, truncated un-modified or modified recombinant collagenases as disclosed herein may be produced by recombinant methods from genetically-modified host cells. Each possibility is a separate embodiment. Any host cell known in the art for the production of recombinant proteins may be used for the present invention. According to some embodiments, the host cell is a prokaryotic cell. Representative, non-limiting examples of appropriate prokaryotic hosts include bacterial cells, such as cells of Escherichia coli and Bacillus subtilis. According to other embodiments, the host cell may be a eukaryotic cell. According to some exemplary embodiments, the host cell may be a fungal cell, such as yeast.

According to some exemplary embodiments, a coding region of interest is a coding region encoding WT-Recombinant collagenase. According to some exemplary embodiments, a coding region of interest is a coding region encoding modified recombinant collagenase.

In some embodiments, the recombinant WT, truncated un-modified or modified recombinant collagenases may be synthesized by expressing a polynucleotide molecule encoding the recombinant collagenase in a host cell, for example, a microorganism cell transformed with the nucleic acid molecule.

According to some embodiments, there is provided a composition which includes a collagenase (recombinant wt, recombinant truncated and/or modified recombinant collagenase polypeptide). In some embodiments, the composition may include one or more suitable excipients, according to the purpose, type and/or use of the composition. In some embodiments, excipient is a pharmaceutical excipient which may include or a pharmaceutical carrier, vehicle, buffer and/or diluent. In some exemplary embodiments, the composition may include carriers (such as, liposomal carriers) harboring or encapsulating the modified recombinant collagenase peptide or nucleic acid encoding the same.

In some embodiments, the administration may be local. According to another embodiment, administration of the composition may be via an injection. For administration via injection, the composition may be formulated in an aqueous solution, for example in a physiologically compatible buffer, or in any suitable carrier, such as, liposomal carriers. Formulations for injection may be presented in unit dosage forms, for example, in ampoules, or in multi-dose containers with, optionally, an added preservative.

According to some embodiments, aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

According to another embodiment, compositions formulated for injection may be in the form of solutions, suspensions, dispersions or emulsions in oily or aqueous vehicles. According to some embodiments, compositions for injection may contain formulator agents such as suspending, stabilizing, and/or dispersing agents.

According to some embodiments, the recombinant WT, truncated un-modified and/or modified recombinant collagenase polypeptide, and/or the composition comprising the same, may be used in combination with other suitable agents. The components of such combinations may be administered sequentially or simultaneously/concomitantly in separate and/or combined formulations by any suitable administration route.

According to some embodiments, there is provided a method of treating or performing an orthodontic procedure, the method may include administration (for example, local administration to an anatomical site) to a subject in need thereof a therapeutically effective amount of a WT, truncated un-modified or modified recombinant collagenases. Each possibility is a separate embodiment. In some embodiments, the modified recombinant collagenase may be administered as a polypeptide as is, or in a suitable composition.

According to some embodiments, the Collagenase is hydrated or dialyzed in PBS. According to some embodiments, the Collagenase are used at concentrations of at least about 4.0 mg/ml.

According to some embodiments, the Collagenase is used at concentrations of between at least about 0.2 mg/ml and about 5.0 mg/ml. According to some embodiments, the Collagenase is used at concentrations of between at least about 0.2 mg/ml and about 10.0 mg/ml.

According to some embodiments, the Collagenase is used at concentrations of between at least about 0.5 mg/ml and about 5.0 mg/ml. According to some embodiments, the Collagenase is used at concentrations of between at least about 1.0 mg/ml and about 5.0 mg/ml.

According to some embodiments, the Collagenase is used at concentrations of between at least about 1.2 mg/ml and about 4.0 mg/ml. According to some embodiments, the Collagenase is used at concentrations of between at least about 1.5 mg/ml and about 4.0 mg/ml. According to some embodiments, the Collagenase is used at concentrations of between at least about 2.5 mg/ml and about 4.0 mg/ml.

According to some embodiments, the Collagenase is used at concentrations of at least about 0.2 mg/ml, at least about 0.5 mg/ml, at least about 0.75 mg/ml, at least about 1.0 mg/ml, at least about 1.25 mg/ml, at least about 1.5 mg/ml, at least about 2.0 mg/ml, at least about 2.5 mg/ml, at least about 3.0 mg/ml, at least about 3.5 mg/ml, at least about 4 mg/ml, at least about 5 mg/ml, or more. Each possibility is a separate embodiment.

According to some embodiments, the Collagenase is used at concentrations of at least about 1.2 mg/ml. According to some embodiments, the Collagenase is used at concentrations of at least about 4 mg/ml.

According to some embodiments, there are provided kits comprising the recombinant un-modified and/or the modified recombinant collagenase peptide and/or the composition as disclosed herein. Each possibility is a separate embodiment. Such a kit may be used, for example, for orthodontic procedures, including, orthodontic tooth movement, prevention of post-orthodontic relapse and tooth alignment.

According to some embodiments, there are provided kits comprising the recombinant WT, recombinant truncated un-modified and/or the truncated modified recombinant collagenase peptide and/or the composition comprising the same as disclosed herein. Each possibility is a separate embodiment.

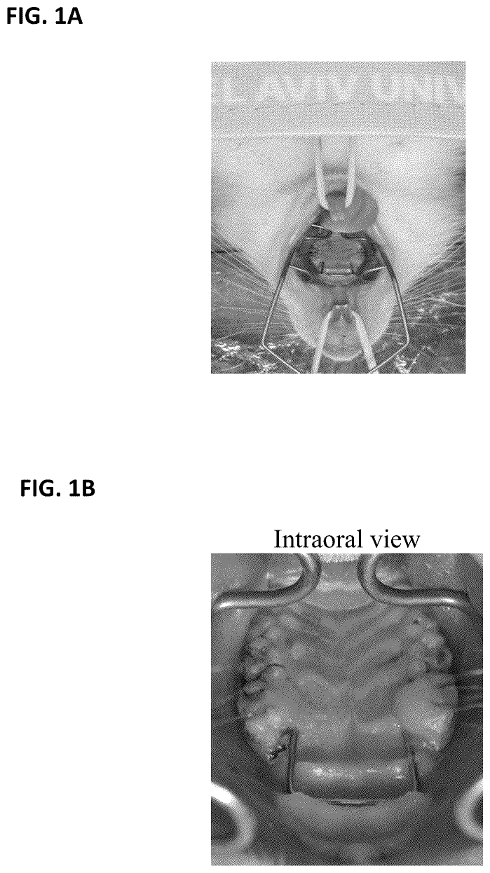

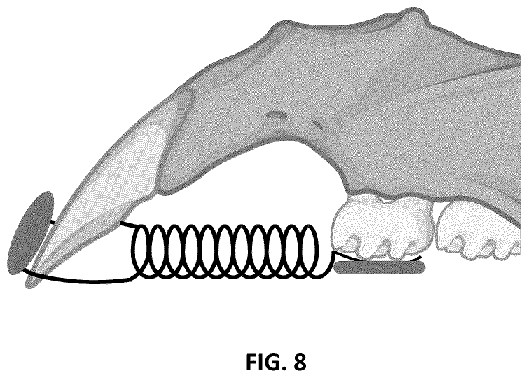

According to some embodiments, the orthodontic procedure comprises applying an appliance designed to apply equal and/or un-equal orthodontic movement on both sides of the upper jaw. Each possibility is a separate embodiment. Reference is made to FIGS. 1A-1B; Example 1 presenting representative images from orthodontic procedure performed for inserting an appliance, and to FIG. 8 illustrating orthodontic procedure performed for applying a closed coil.

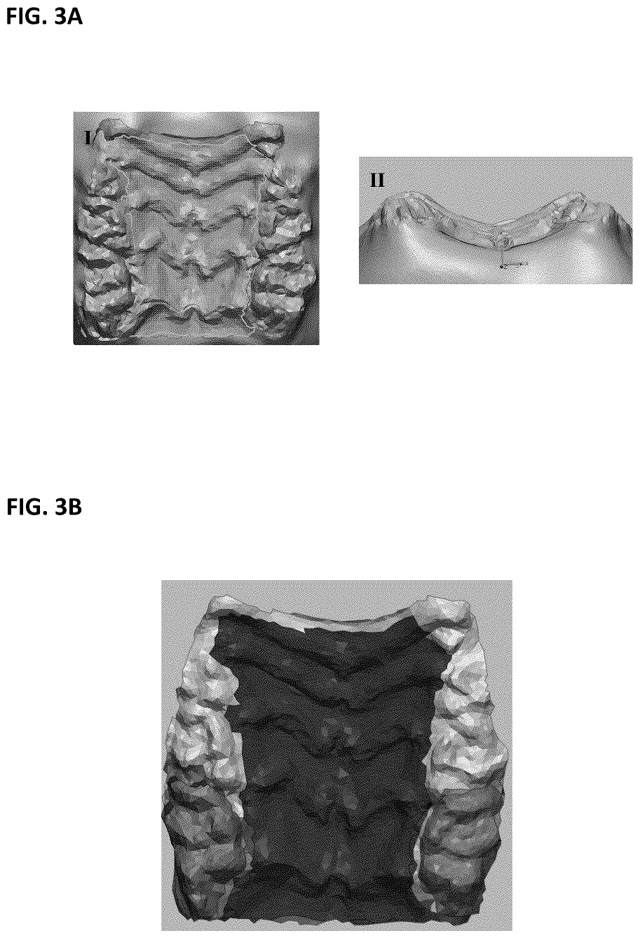

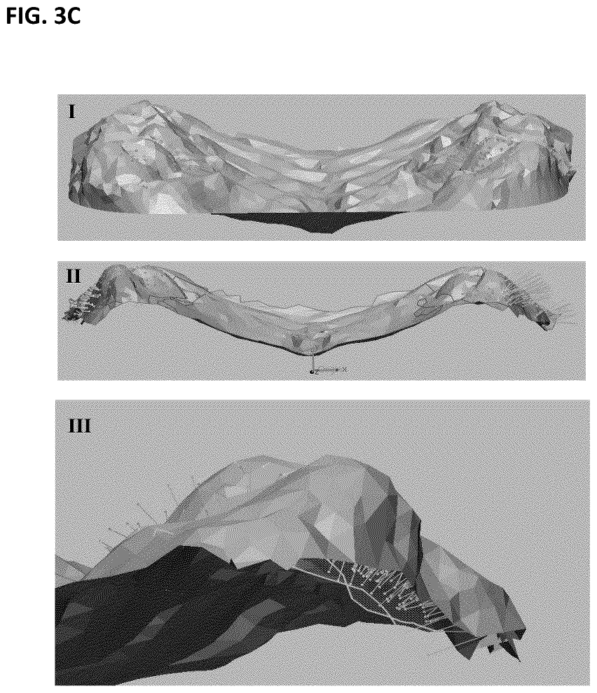

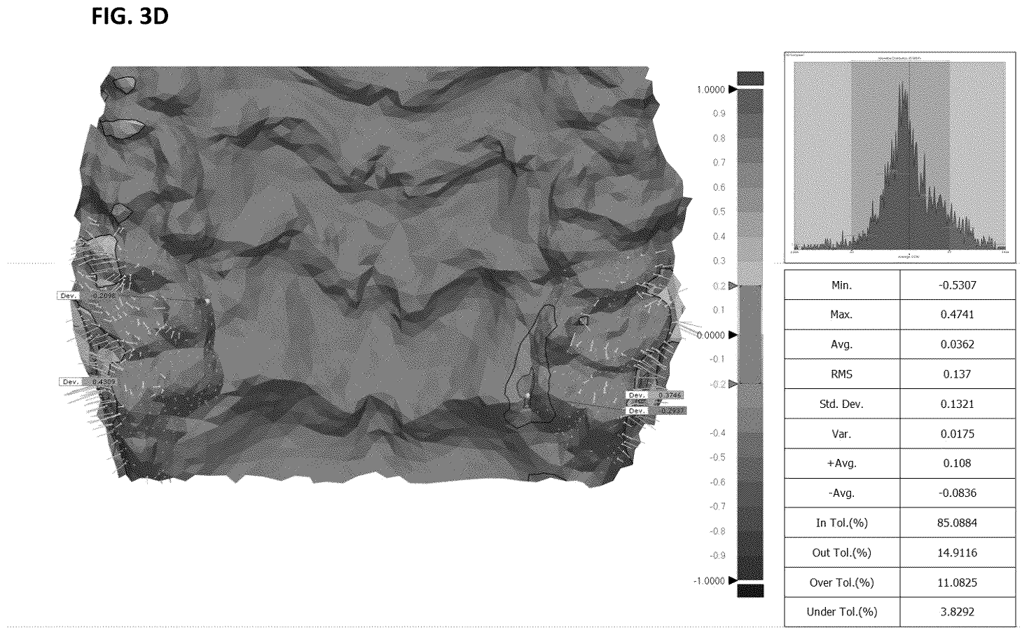

According to some embodiments, the orthodontic procedure comprises measurements of tooth movement and/or post-orthodontic relapse. Each possibility is a separate embodiment. Reference is made to FIGS. 3A-3E and Example 1 illustrating and demonstrating calculation of deviations in tooth movement performed utilizing superimpositions of intraoral digital scans based on a reference structure (the palate) by a computer software.

According to some embodiments, the polypeptide or a composition including the same may be administered prior to, during or after the orthodontic procedure. For example, the polypeptide or the composition may be administered 1-28 days, or any sub-range therein, prior to or after the orthodontic procedure (for example, tooth movement or prevention of relapse). For example, the polypeptide or the composition may be administered 1-24 hours minutes prior to or after the procedure. For example, the polypeptide or the composition may be administered 1-96 hours minutes prior to or after the procedure. For example, the polypeptide or the composition may be administered 1-48 hours minutes prior to or after the procedure. For example, the polypeptide or the composition may be administered 1-360 minutes prior to or after the procedure.

According to some embodiments, the polypeptide or the composition may be administered at the initiation of the treatment (for example, 1-48 hours) after placement of an orthodontic appliance, during the treatment (for any time period needed to accelerate tooth movement for ex. orthodontic mechanics stage) and/or before the end of the procedure (for example, any time which is more than 1 week before the removal of the orthodontic appliance).

According to some embodiments, as exemplified herein, there is provided a recombinant collagenase enzyme-based treatment for use in minimally invasive orthodontic procedures.

According to some embodiments, as exemplified herein, enzymatic disruption (utilizing recombinant collagenase and/or modified recombinant collagenase) of the connective tissue of the marginal gingiva (lamina propria), can be used to degrade the gingival fibers, thereby preventing or reducing relapse after orthodontic tooth movement.

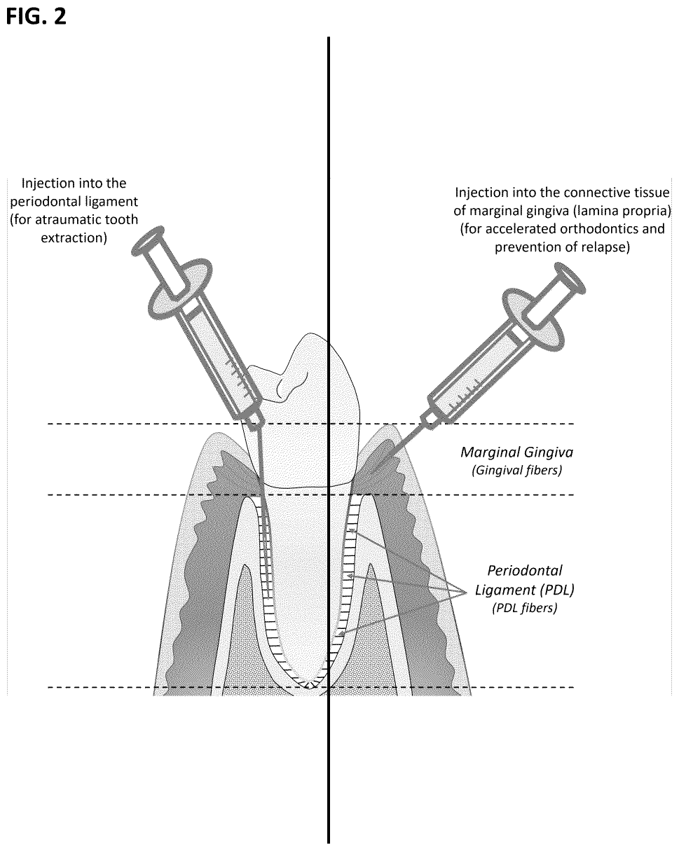

Reference is now made to FIG. 2, which shows an illustration of a tooth and the administration location of collagenases (or compositions including the same) when used for orthodontic procedures as exemplified in Example 1 and Example 2 as well as in Example 3.

As shown in FIG. 2, in contrast to localized administration to the PDL region (for example, for a-traumatic tooth extraction), for orthodontic procedures, the herein disclosed localized administration is advantageously performed into the connective tissue of the laminal propria (marginal gingiva). The two regions are substantially different with respect of accessibility and compositions of fibers residing in each region. As is known in the art, Dentino-gingival and transseptal fiber groups constitute the gingival fiber system, while the PDL fiber groups are classified as horizontal, oblique, and apical. Thus, in contrast to application of collagenase for atraumatic tooth extraction, that is injected into the PDL space (between the root cementum and the alveolar bone), in order to degrade PDL fibers, for orthodontic procedures (for example, “enzymatic fiberotomy”), the collagenase composition is injected into the connective tissue of the marginal gingiva (called lamina propria), in order to degrade the gingival fibers, as illustrated in FIG. 2 (Right side of the illustration). Periodontal ligament and gingival fiber groups are composed mostly of collagen type-I and are situated in proximity, however, several functional differences are known: gingival fibers bundles are not readily rearranged after orthodontic tooth movement (“OTM”), leading to a more persistent effect from the gingival fibers than from the PDL fibers on relapse after orthodontic tooth movement. Moreover, gingival fibers are of higher density than the PDL fibers, leading to a strain in the gingival fibers, which persists even after a retention period. In contrast to the PDL fibers, the lower turnover rate of the gingival fibers renders them stretched and un-remodeled for months after OTM, leading to a long-term relapse. Hence, localized administration into that region of the collagenase composition disclosed herein (i.e., including WT, truncated and/or modified) can advantageously result in acceleration of tooth movement and prevention or at least partial reduction of long-term relapse.

Thus, according to some embodiments, as exemplified herein, in contrast to the surgical fiberotomy, the enzymatic fiberotomy as disclosed herein, is accomplished in a minimally invasive manner, without surgical intervention that can compromise healthy tissues. Surgical fiberotomy also called circumferential supra-crestal fiberotomy (CSF), may include cutting the supra-alveolar gingival fibers (dentogingival and dentoperiosteal). Additionally, enzymatic fiberotomy can expand the therapeutic spectrum for patients with coagulation problems and prevent complications such as discomfort and post-operative pain.

According to some aspects, there is provided a method of performing an orthodontic procedure in a subject in need thereof, the method comprising administering to the subject in need thereof an effective amount of a recombinant collagenase polypeptide, or a composition comprising the same.

In the description and claims of the application, the words “include” and “have”, and forms thereof, are not limited to members in a list with which the words may be associated. As used herein, the term comprising includes the term consisting of.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1—Enzymatic Fiberotomy with Truncated Recombinant Collagenase (ColG) Showed an Improved Effect on the Degree of Orthodontic Tooth Movement Relative to Surgical Fiberotomy

### Material and Methods

The following description of the materials and methods applies for both Example 1 and Example 2.

N-terminally truncated recombinant collagenase G (ColG) derived from Clostridium histolyticum—A polynucleotide sequence of the coding sequence of a WT Collagenase G protein from Clostridium histolyticum missing the first 118 amino acid residues (i.e., N-terminal truncation), comprising residues Tyr119-Lys1118 with respect to a full length WT Collagenase G protein, was cloned into a pET15b plasmid with an N-terminus HisX6-tag followed by a tobacco etch virus (TEV) cleavage site, thereby creating a truncated recombinant form of a WT collagenase G (ColG) that was derived from Clostridium histolyticum.

The full-length WT Collagenase G protein from Clostridium histolyticum (residues 1-1118) has the amino acid sequence as denoted by SEQ ID NO: 8 below:

MKKNILKILMDSYSKESKIQTVRRVTSVSLLAVYLTMNTSSLVLA
KPIENTNDTSIKNVEKLRNAPNEENSKKVEDSKNDKVEHVKNIEE
AKVEQVAPEVKSKSTLRSASIANTNSEKYDFEYLNGLSYTELTNL
IKNIKWNQINGLFNYSTGSQKFFGDKNRVQAIINALQESGRTYTA
NDMKGIETFTEVLRAGFYLGYYNDGLSYLNDRNFQDKCIPAMIAI
QKNPNFKLGTAVQDEVITSLGKLIGNASANAEVVNNCVPVLKQFR
ENLNQYAPDYVKGTAVNELIKGIEFDFSGAAYEKDVKTMPWYGKI
DPFINELKALGLYGNITSATEWASDVGIYYLSKFGLYSTNRNDIV
QSLEKAVDMYKYGKIAFVAMERITWDYDGIGSNGKKVDHDKFLDD
AEKHYLPKTYTEDNGTFIIRAGDKVSEEKIKRLYWASREVKSQFH
RVVGNDKALEVGNADDVLTMKIENSPEEYKENTNINGVSTDNGGL
YIEPRGTFYTYERTPQQSIFSLEELFRHEYTHYLQARYLVDGLWG
QGPFYEKNRLTWFDEGTAEFFAGSTRTSGVLPRKSILGYLAKDKV
DHRYSLKKTLNSGYDDSDWMFYNYGFAVAHYLYEKDMPTFIKMNK
AILNTDVKSYDEIIKKLSDDANKNTEYQNHIQELADKYQGAGIPL
VSDDYLKDHGYKKASEVYSEISKAASLTNTSVTAEKSQYFNTFTL
RGTYTGETSKGEFKDWDEMSKKLDGTLESLAKNSWSGYKTLTAYF
TNYRVTSDNKVQYDVVFHGVLTDNADISNNKAPIAKVTGPSTGAV
GRNIEFSGKDSKDEDGKIVSYDWDFGDGATSRGKNSVHAYKKAGT
YNVTLKVTDDKGATATESFTIEIKNEDTTTPITKEMEPNDDIKEA
NGPIVEGVTVKGDLNGSDDADTFYFDVKEDGDVTIELPYSGSSNF
TWLVYKEGDDQNHIASGIDKNNSKVGTFKSTKGRHYVFIYKHDSA
SNISYSLNIKGLGNEKLKEKENNDSSDKATVIPNFNTTMQGSLLG
DDSRDYYSFEVKEEGEVNIELDKKDEFGVTWTLHPESNINDRITY
GQVDGNKVSNKVKLRPGKYYLLVYKYSGSGNYELRVNK

The amino acid sequence of the N-terminally truncated recombinant collagenase polypeptide (a.a 119-1118)—is denoted by SEQ ID NO: 9 below:

YDFEYLNGLSYTELTNLIKNIKWNQINGLFNYSTGSQKFFGDKNR
VQAIINALQESGRTYTANDMKGIETFTEVLRAGFYLGYYNDGLSY
LNDRNFQDKCIPAMIAIQKNPNFKLGTAVQDEVITSLGKLIGNAS
ANAEVVNNCVPVLKQFRENLNQYAPDYVKGTAVNELIKGIEFDFS
GAAYEKDVKTMPWYGKIDPFINELKALGLYGNITSATEWASDVGI
YYLSKFGLYSTNRNDIVQSLEKAVDMYKYGKIAFVAMERITWDYD
GIGSNGKKVDHDKELDDAEKHYLPKTYTFDNGTFIIRAGDKVSEE
KIKRLYWASREVKSQFHRVVGNDKALEVGNADDVLTMKIFNSPEE
YKENTNINGVSTDNGGLYIEPRGTFYTYERTPQQSIFSLEELFRH
EYTHYLQARYLVDGLWGQGPFYEKNRLTWFDEGTAEFFAGSTRTS
GVLPRKSILGYLAKDKVDHRYSLKKTLNSGYDDSDWMFYNYGFAV
AHYLYEKDMPTFIKMNKAILNTDVKSYDEIIKKLSDDANKNTEYQ
NHIQELADKYQGAGIPLVSDDYLKDHGYKKASEVYSEISKAASLT
NTSVTAEKSQYFNTFTLRGTYTGETSKGEFKDWDEMSKKLDGTLE
SLAKNSWSGYKTLTAYFTNYRVTSDNKVQYDVVFHGVLTDNADIS
NNKAPIAKVTGPSTGAVGRNIEFSGKDSKDEDGKIVSYDWDFGDG
ATSRGKNSVHAYKKAGTYNVTLKVTDDKGATATESFTIEIKNEDT
TTPITKEMEPNDDIKEANGPIVEGVTVKGDLNGSDDADTFYFDVK
EDGDVTIELPYSGSSNFTWLVYKEGDDQNHIASGIDKNNSKVGTF
KSTKGRHYVFIYKHDSASNISYSLNIKGLGNEKLKEKENNDSSDK
ATVIPNENTTMQGSLLGDDSRDYYSFEVKEEGEVNIELDKKDEFG
VTWTLHPESNINDRITYGQVDGNKVSNKVKLRPGKYYLLVYKYSG
SGNYELRVNK

The amino acid sequence of the N-terminally truncated recombinant collagenase polypeptide (ColG) fused to His-tag (HHHHHH) followed by a tobacco etch virus (TEV) cleavage site (NLYFO) at the N-terminus-is denoted by SEQ ID NO: 1 below:

MGSSHHHHHHSSGENLYFQGGTMYDFEYLNGLSYTELTNLIKNIKW
NQINGLENYSTGSQKFFGDKNRVQAIINALQESGRTYTANDMKGI
ETFTEVLRAGFYLGYYNDGLSYLNDRNFQDKCIPAMIAIQKNPNF
KLGTAVQDEVITSLGKLIGNASANAEVVNNCVPVLKQFRENLNQY
APDYVKGTAVNELIKGIEFDFSGAAYEKDVKTMPWYGKIDPFINE
LKALGLYGNITSATEWASDVGIYYLSKFGLYSTNRNDIVQSLEKA
VDMYKYGKIAFVAMERITWDYDGIGSNGKKVDHDKFLDDAEKHYL
PKTYTEDNGTFIIRAGDKVSEEKIKRLYWASREVKSQFHRVVGND
KALEVGNADDVLTMKIFNSPEEYKFNTNINGVSTDNGGLYIEPRG
TFYTYERTPQQSIFSLEELFRHEYTHYLQARYLVDGLWGQGPFYE
KNRLTWFDEGTAEFFAGSTRTSGVLPRKLILGYLAKDKVDHRYSL
KKTLNSGYDDSDWMFYNYGFAVAHYLYEKDMPTFIKMNKAILNTD
VKSYDEIIKKLSDDANKNTEYQNHIQELVDKYQGAGIPLVSDDYL
KDHGYKKASEVYSEISKAASLTNTSVTAEKSQYFNTFTLRGTYTG
ETSKGEFKDWDEMSKKLDGTLESLAKNSWSGYKTLTAYFTNYRVT
SDNKVQYDVVFHGVLTDNGDISNNKAPIAKVTGPSTGAVGRNIEF
SGKDSKDEDGKIVSYDWDFGDGATSRGKNSVHAYKKAGTYNVTLK
VTDDKGATATESFTIEIKNEDTTTPITKEMEPNDDIKEANGPIVE
GVTVKGDLNGSDDADTFYFDVKEDGDVTIELPYSGSSNFTWLVYK
EGDDQNHIASGIDKNNSKVGTFKATKGRHYVFIYKHDSASNISYS
LNIKGLGNEKLKEKENNDSSDKATVIPNFNTTMQGSLLGDDSRDY
YSFEVKEEGEVNIELDKKDEFGVTWTLHPESNINDRITYGQVDGN
KVSNKVKLRPGKYYLLVYKYSGSGNYELRVNK

Expression and purification of a truncated recombinant collagenase G (ColG)—The amino acid sequence of the N-terminally truncated recombinant collagenase polypeptide (ColG) fused to His-tag (HHHHHH) followed by a tobacco etch virus (TEV) cleavage site (NLYFO) at the N-terminus—as denoted by SEQ ID NO: 1—was cloned into a pET15b plasmid and transformed into E. coli competent cells. Selected colonies were transferred to LB medium and ColG protein expression was induced using 1 mM IPTG for 16 h at 25° C. Next, cells were harvested, and the recombinant ColG protein was purified using immobilized metal affinity chromatography (IMC), followed by ion exchange chromatography (IEC).

Ethics—the experiments disclosed herein were carried out in accordance with the Helsinki Accord at the animal care unit of the Faculty of Medicine, Tel Aviv University (TAU-MD-IL-2036-140-3).

In vivo experimental set-up—24 female Sprague Dawley rats, 4-month-old, 200-250 gram weight were divided into five groups. The rats were acclimatized for 1 week before the start of the experiment. On the day of the orthodontic procedure, or at the time of measuring tooth movement, the rats were anesthetized using a mixture of 25 mg/kg body weight of ketamine HCl and 42 mg/kg body weight of xylazine hydrochloride by intramuscular injection (IM).

A case crossover (split-mouth) design was applied in this study, where one side of the mouth received the treatment of the orthodontic procedure (i.e., surgical fiberotomy or the injection of collagenase) and the other served as a control as demonstrated in FIGS. 1A-1B:
Group A: five rats, split-mouth design, enzymatic fiberotomy using recombinant collagenase (ColG) (SEQ ID NO: 1) was performed on the first molar on one side, while the contralateral side served as a control (no treatment).
Group B: five rats, split-mouth design, surgical fiberotomy using a scalpel was performed on the first molar on one side, while the contralateral side served as a control (no treatment).
Group C: five rats, spilt-mouth design, an enzymatic fiberotomy using recombinant collagenase (ColG) (SEQ ID NO: 1) was performed on the first molar on one side, and surgical fiberotomy using a scalpel was performed on the contralateral side.
Group D: five rats, split-mouth design, enzymatic fiberotomy using commercial collagenase (C.COL) was performed on the first molar on one side, while the contralateral side served as a control (no treatment).
Group E: four rats, spilt-mouth design. To examine whether the injection per se would provoke an effect, Dulbecco's Phosphate Buffer Saline (PBS) was injected into the first molar on one side; the contralateral side served as a control (no treatment).

Orthodontic procedure—The appliance was designed to apply equal orthodontic movement on both sides of the upper jaw using a 0.012 nickel-titanium (NiTi) wire, that was cinched and inserted into the embrasures between the first and second molars. The wire was bonded to the palatal side of the first molars with flowable composite (Flow-It ALC, Pentron) (as shown in FIGS. 1A-1B). Orthodontic force was applied for 28 days, followed by 14 days (up to day 42) without force to observe and measure relapse. These time intervals have been shown to be sufficient for significant changes.

Injection of collagenase to the lamina propria of the marginal gingiva—As shown in FIG. 2 (Right side) Collagenase or PBS were injected into the lamina propria of the marginal gingiva using a computer-assisted injection system (Wand Single Tooth Anesthesia system, Milestone Scientific, Roseland, NJ, USA), which enabled controllable and precise injection through a clinically accepted approach. Standard cartridges containing the local anesthetic solution for dental injection were accurately emptied of their content, washed with PBS and filled with either collagenase or PBS solution. Injection was performed using a 30 G 2.54 cm length needle inserted into the marginal gingiva. The injection was repeated at four sites around the molars: on the buccal, lingual, mesial, and distal sites, in a total volume of 50 μl. Collagenase was used at a concentration of 4 mg/ml, which was hydrated or dialyzed in PBS.

Fiberotomy procedure—Circumferential fiberotomy was made using a 11C scalpel inserted between the free gingiva and the tooth until reaching the bone level.

Measurement procedure—measurements of OTM were performed at day 0 and 28, and post-orthodontic relapse was measured at days 35 and/or 42, as shown in FIGS. 3A-3D. The analysis of OTM (measured at day 0, 28, presented as T₀-T₂₈) and post-orthodontic relapse (measured at day 35, 42, presented as a ratio T35/T28, T42/T28. Reference is made to Example 2) was addressed using an intra-oral scanner (TRIOS 5 from 3Shape, Copenhagen, Denmark). The measurements were performed on the 3D intraoral scans of the maxilla, using Geomagic Control X software (Geomagic U.S., Research Triangle Park, NC).

First, the intraoral scans were aligned using 3D surface-based registration (based on the surface area to fit the preoperative and postoperative models). After the scans were aligned, they were best fitted based on segmentation of the palate as a reference, which has been shown to have a more accurate superimposition than the mandible, moreover, it was proven to be a stable region of interest during growth and unchanged by bone modeling associated with OTM (FIG. 3A). Best fitting was performed using global and fine regional surface registration (the program automatically performed fine-tuned automatic adjustments to the spatial position of the two models based on all points and changed the coordinates of one object to match the other using a best-fit surface algorithm). This technique of ‘fine matching’ uses thousands of reference points instead of a few landmarks/areas and is based on ‘Iterative Closest Point Algorithms’ (ICP).

Once the scans were aligned, both molars were segmented, outlined, and 3D compared to check for deviations from the reference scan (FIGS. 3B-3D).

Statistics—Statistical analysis was performed using the GraphPad Prism software version 9.5. The mean and standard deviation (SD) of first molar tooth movements in the in vivo experiment for the OTM during the activation period were statistically analyzed for changes over time within the same group using a paired t-test using a two-tail test, while the relapse was analyzed using matched two-way ANOVA with Tukey multiple correction, comparing both molars in each rat owing to the split-mouth nature of the study. Statistical significance was set at p<0.05.

### Results

Weight of the animals—in all five groups, the weight of the rats decreased during the 28 days of active phase. The weight reduction did not exceed 10% of the initial weight. After the orthodontic appliance was removed (day 28), the rats regained most of their body weight. There were no significant differences in the weight of the rats among the groups.

Tooth movement—Displacement of the first maxillary molars was measured in rats after activation of the orthodontic appliance (T₀-T₂₈) as described in the materials and methods.

As can be seen in FIGS. 4A-4E, for all five treatment groups, the movement of the first molar on the treated side was greater than that on the control side, except for the PBS group (Group E; FIG. 4A), which showed greater movement on the control side, with a mean difference of 0.092+0.008 mm (p<0.01).

In the commercial collagenase group (C.COL) (Group D; FIG. 4E), a mean difference of 0.016+0.029 mm was recorded (p<0.46) for the movement of the first molar on the treated side relative to that of the control side, suggesting treatment with C.COL did not facilitate significant tooth movement after orthodontic appliance.

Differently, in the surgical group (Group B; FIG. 4B), tooth movement of the first molar on the treated side was significantly greater (p<0.05) than tooth movement on the control side with mean differences of 0.062+0.032.

Advantageously and surprisingly, in the recombinant collagenase (COLG) group (Group A; FIG. 4C), tooth movement of the first molar on the treated side was also significantly greater (p<0.05) than tooth movement on the control side with mean difference of 0.057+0.028 mm, suggesting that treatment with COLG is equally effective as traditional surgery, only without the complications and damage associated with the surgery.

Even more advantageous and surprising, was that rats that underwent enzymatic fiberotomy using COLG on one side and surgical fiberotomy on the other side (Group C; FIG. 4D) showed significantly greater tooth movement of the first molar where COLG was injected (p<0.05) relative to the side where surgical fiberotomy was performed, with a mean difference of 0.07±0.029 mm, suggesting that treatment with COLG may be more effective than traditional surgery.

In order to show the change in percentage calculations were made by dividing the OTM of the test side with the standard deviation of the test side, and the OTM of the control side with the standard deviation of the control side, and calculating the ratio between them and by multiplying by 100.

When considering such calculations the results indicate that PBS reduced orthodontic movement by a mean of 49.4±6.7% (FIG. 4A), whereas surgical (FIG. 4B) and enzymatic fiberotomy with C.COL (FIG. 4E) and COLG (FIG. 4C) increased orthodontic movement by a mean of 54.7±30.8%, 14.2±28% and 34.3±32.4%, respectively. Moreover, when directly comparing enzymatic fiberotomy using COLG to surgical fiberotomy (FIG. 4D), COLG showed enhanced orthodontic movement by a mean of 32.3±18.3%, in some embodiments.

To conclude, when comparing enzymatic fiberotomy using COLG to surgical fiberotomy, COLG showed at least a similar potential, and possibly even an enhanced ability for acceleration of tooth movement.

### Example 2—Enzymatic Fiberotomy with Truncated Recombinant Collagenase (ColG) and Surgical Fiberotomy Showed Improved Effect on the Degree of Post-Orthodontic Relapse

### Material and Methods

Post-orthodontic relapse was assessed using the same experimental set-up and during the same experiments performed to assess orthodontic tooth movement (OTM), therefore, reference is now made to the materials and methods of Example 1.

### Results

Relapse—The displacement ratio of the first maxillary molars from T28 was recorded after removal of the orthodontic appliance after one and two weeks (week 1: T28-T35; week 2: T35-T42) (Table 1), and was calculated as described in the material and methods of Example 1 and illustrated in FIGS. 3A-3D. The results are presented in FIGS. 5A-5E and are summarized in Table 1 below, presenting the cumulative relapse that occurred after deactivation at T28 which is the time when the appliances were removed, and the teeth started to return to their original position (relapse), and the measurements commenced (from T28). Relapse is a ratio entity. The relapse was calculated as the ratio of the change between the scans at T35 and T42 (each measured relative to T28 (the deactivation phase).

TABLE 1
Orthodontic relapse ratio from T28, in a rat model

| | | Orthodontic relapse | | | |
|---|---|---|---|---|---|
| | | | Control | Difference | |
| | | Test side/T₂₈ | side^{†}/T₂₈ | Test-Control | |
| Group | | Mean | Mean | Mean | SE |
| PBS- | Week 1 | 0.8 | 0.47 | 0.33 | 0.1 |
| Control | Week 2 | 1.0 | 0.76 | 0.24 | |
| Surgical- | Week 1 | 0.38 | 0.57 | −0.19* | 0.04 |
| Control | Week 2 | 0.62* | 0.86* | −0.24* | |
| C. COL- | Week 1 | 0.28 | 0.49 | −0.21 | 0.03 |
| Control | Week 2 | 0.62* | 0.94* | −0.32* | |
| COLG- | Week 1 | 0.42 | 0.64 | −0.22** | 0.01 |
| Control | Week 2 | 0.68** | 0.94** | −0.26** | |
| COLG- | Week 1 | 0.37 | 0.42 | −0.05 | 0.03 |
| Surgical | Week 2 | 0.56* | 0.66* | −0.1 | |

^{†}In the COLG-surgical group, the control side was the surgical group.
*p < 0.05,
**p < 0.01

As can be seen in FIGS. 5A-5E and in Table 1, for all five treatment groups, the relapse ratio of the first molar on the treated side was smaller than that on the control side, except for the PBS group (Group E; FIG. 5A), which showed greater relapse on the PBS injected treated side with a mean ratio difference of 0.33±0.1 (p<0.2) at week 1 and 0.24±0.1 (p<0.32) at week 2.

Also, in all groups except the PBS group, there was a significant difference between the relapse that occurred in the first week and that of the second week (p<0.05, p<0.01 in the COLG group).

Advantageously and unexpectedly, both the surgical group (Group B; FIG. 5B) and the COLG group (Group A; FIG. 5C) showed a significant reduction in relapse at weeks 1 and 2. The surgical group showed a mean ratio difference of 0.19±0.04 at week 1 (p<0.05) and 0.24±0.04 (p<0.05) at week 2. While the COLG group had a mean ratio difference of 0.22±0.01 (p<0.01) at week 1 and 0.26±0.01 (p<0.01) at week 2 (Table 1), in some embodiments.

This indicates that treatment with COLG provides at least similar, and possibly even improved, reduction in relapse to the reduction provided by the surgical fiberotomy treatment.

Moreover, this conclusion is also supported by the similar mean ratio difference that was calculated in rats for which surgical fiberotomy treatment was performed on one side and enzymatic fiberotomy treatment with COLG was performed on the other side (Group C; FIG. 5D). This group showed a similar effect on the degree of post-orthodontic relapse both at week 1 and at week 2 with a mean ratio difference of 0.05±0.03 (p<0.47) and 0.1±0.03 (p<0.15) at weeks 1 and 2.

Finaly, C.COL (Group D; FIG. 5E), which didn't have a significant effect during the active phase, didn't have one in the first week of relapse as well, with a mean ratio difference of 0.21±0.03 (p<0.056), however, in the second week of relapse the effect was significant with a mean ratio difference of 0.32±0.03 (p<0.05).

### Example 3—Assessment of the Effect of Enzymatic Fiberotomy with Modified Recombinant Collagenase on the Degree of Orthodontic Tooth Movement and Post-Orthodontic Relapse

### Material and Methods

N-terminally truncated modified recombinant collagenase—modified collagenases containing point mutations were generated from the un-modified N-terminally truncated collagenases having the amino acid sequence as denoted by SEQ ID NO: 1 or SEQ ID NO: 9, thereby creating N-terminally truncated mutated/modified recombinant collagenases. Polynucleotide sequences of the coding sequence of the N-terminally truncated mutated/modified recombinant collagenases were cloned into a pET15b plasmid with an N-terminus HisX6-tag followed by a tobacco etch virus (TEV) cleavage site.

These modified collagenases containing point mutations were generated using standard genetic engineering techniques, to create a thermos-stable and active enzymes. PROSS algorithm (Goldenzweig, A. et al. Automated Structure- and Sequence-Based Design of Proteins for High Bacterial Expression and Stability. Mol. Cell 2016, 63 (2), 337-346) was utilized. Since ColG Xray structure lacks the Ca2+ ion, while a water molecule is located in its cavity, the water was replaced with a Ca2+ Ion.

The amino acid sequences of the N-terminally truncated modified recombinant collagenases are denoted by any one of SEQ ID NOs: 2-4 (or SEQ ID NOs: 5-7, respectively) below:

Modified recombinant collagenase polypeptide
(also referred to as Des1)-
SEQ ID NO: 2
YDFEYLNGLSYTELTNLIKNIKWNQINGLFQYSTGSQKFFGDKNR
VQAIINALQESGRTYTANDDKGIETFTEVLRAGFYLGYYYDGLSY
LNDRNFQDKCIPAMIAIQKNPNFKLGTAVQDEVITSLGKLIGNAS
ANAEVVNNCVPVLKQFRENLNQYAPDYVKGTAVYELIKGIEYDES
GAAYEKDVKTMPWYGKIDPFINELKALGLYGNITSDTEWASDVGI
YYLSKFGLYHTNRNDIVQSLEKAVDMYKYGKIAFVAMERITWDYD
GIGSNGKKVDHDKFLDDAEKHYLPKTYTFDNGTFIIRAGDKVSEE
KIKRLYWASREVKSQFHRVVGNDKPLEVGNADDVLTMKIFNSPEE
YKENTNINGVSTDNGGLYIEPRGTFYTYERTPQQSIFSLEELFRH
EYTHYLQARYLVPGLWGQGPFYEKNRLTWFDEGTAEFFAGSTRTS
GVLPRKLILGYLAKDKVDHRYSLKKTLNSGYDDSDWMFYNYGFAV
AHYLYEKDMPTFIKMNKAILNNDVKSYDEIIKKLSDDANKNTEYQ
NHIQELVDKYDNATIPLVSDDYLKDHGYKKASEVYSEISKAANLT
NTSVTEHKSQYFNTFTLRGTYTGETSKGEFKDWKEMSKKLDGTLE
SLAKNSWSGYKTLTAYFTNYRVTSDNKVQYDVVFHGVLTDNGDIS
NNKAPIAKVTGPSTGAVGRNIEFSGKDSKDEDGKIVSYDWDFGDG
ATSRGKNSVHAYKKAGTYNVTLKVTDDKGATATESFTIEIKNEDT
TTPITKEMEPNDDIKEANGPIVEGVTVKGDLNGSDDADTFYFDVK
EDGDVTIELPYSGSSNFTWLVYKEGDDQNHIASGIDKNNSKVGTF
KATKGRHYVFIYKHDSASNISYSLNIKGLGNEKLKEKENNDSSDK
ATVIPNFNTTMQGSLLGDDSRDYYSFEVKEEGEVNIELDKKDEFG
VTWTLHPESNINDRITYGQVDGNKVSNKVKLRPGKYYLLVYKYSG
SGNYELRVNK-
Modified recombinant collagenase polypeptide
(also referred to as Des4)-
SEQ ID NO: 3
YDFEYLNGLSYTELINLIKNIKWNQINGLFQYSDGSQKFYGDKNR
VQAIINALQESGRTYTANDDKGIETFTEVLRAGFYLGYYYDGLSY
LNDRNFQDKCIPAMIAIQKNPNFKLGTAVQDEVIASLGKLIGNAS
ANAEVVNNCVPVLKQFRENLNQYAPDYVKGTAVYELIKGIEYDES
GAAYEKDVKTMPWYGKIDPFINELKKLGLYGNITSDTEWASDVGI
YYLSKFGLYHTNRNDIVQALEKAVDMYKYGKIAFVAMERIKWDYD
GIGSNGKKVDHDKFLEDAEKHYLPKTYTEDNGTFIIRAGDKVSEE
KIKRLYWASKEVKAQFHRVVGNDKPLEVGNADDVLTMKIYNSPEE
YKENTYINGVSTDNGGLYIEPRGTFYTYERTPQQSIFSLEELFRH
EYTHYLQARYLVPGLWGQGPFYENNRLTWFDEGTAEFFAGSTRTS
GVLPRKTILGYLAKDKVDHRYSLKKTLNSGYDDSDWMFYNYGFAV
AHYLYEKDMPTFIKMHKAILNNDVKSYDEIIKKLSDDANKNKEYQ
NHIQELVDRYDNATIPLVSDDYLKDHGYKPASEVYSEISKAANLT
NTSVTKHKSQFFNTFTLRGTYTGGTSKGEFKDWKEMSKKLDETLE
SLSKKSWSGYKTLTAYFTNYRVTSDNKVQYDVVFHGVLTDNGDIS
NNKAPIAKVTGPSTGAVGRNIEFSGKDSKDEDGKIVSYDWDFGDG
ATSRGKNSVHAYKKAGTYNVTLKVTDDKGATATESFTIEIKNEDT
TTPITKEMEPNDDIKEANGPIVEGVTVKGDLNGSDDADTFYFDVK
EDGDVTIELPYSGSSNFTWLVYKEGDDQNHIASGIDKNNSKVGTF
KATKGRHYVFIYKHDSASNISYSLNIKGLGNEKLKEKENNDSSDK
ATVIPNFNTTMQGSLLGDDSRDYYSFEVKEEGEVNIELDKKDEFG
VTWTLHPESNINDRITYGQVDGNKVSNKVKLRPGKYYLLVYKYSG
SGNYELRVNK
Modified recombinant collagenase polypeptide
(also referred to as Des6)-
SEQ ID NO: 4
YDFEYLNGLSYDELTNLIKNIKWNQINGLFQYSDGSQKFYGDKNR
VQAIINALEESGRTYTANDDKGIETFTEVLRAGFYLGYYYDGLSY
LNDRNFQDKCIPAMIAIQKNPNFKLGTDVQDEVIAALGKLIGNAS
ANAEVVNNCVPVLKQFRENLNQYAPDYSKGTAVYELIKGIEYDFS
GAAYEKDPKTMPWYGKIDPFINELKKLGLYGNITSDTEWASNVGI
YYLSKFGKYHSNRNDIVQALEKAVDMYKYGKIAFVAMERIKEDYD
GIGSNGKKVDHDKFKEDAEKHYLPKTYTFDNGTFIIRAGDKVSEE
KIKRLYWASKEVKAQFHRVVGNDKPLEVGNADDVLTMKIYNSPEE
YKENTYINGVSTDNGGIYIEPRGTFYTYERTPQQSIFSLEELFRH
EFTHYLQARYLVPGLWGQGPFYENNRLTWFDEGTAEFFAGSTRTS
GVLPRKTILGYLAKDKVDHRYSLKKTLNSGYDDSDWMFYNYGFAV
AHYLYEKDMPTFIKMHKAILNNDVKSYDEYIKKLSDDANKNKEYQ
NHIQELVDRYDNATIPLVSDDYLKDHGYKPASEIYSEIAKAANLT
NTSVTKHKSQFFNTFTLRGTYTGGTSKGEFQDWKEMNKKLDEILE
QLSKKSWSGYKTLTAYFTNYRVTSDNQVQYDVVFHGVLTDNGDIS
NNKAPIAKVTGPSTGAVGRNIEFSGKDSKDEDGKIVSYDWDFGDG
ATSRGKNSVHAYKKAGTYNVTLKVTDDKGATATESFTIEIKNEDT
TTPITKEMEPNDDIKEANGPIVEGVTVKGDLNGSDDADTFYFDVK
EDGDVTIELPYSGSSNFTWLVYKEGDDQNHIASGIDKNNSKVGTF
KATKGRHYVFIYKHDSASNISYSLNIKGLGNEKLKEKENNDSSDK
ATVIPNFNTTMQGSLLGDDSRDYYSFEVKEEGEVNIELDKKDEFG
VTWTLHPESNINDRITYGQVDGNKVSNKVKLRPGKYYLLVYKYSG
SGNYELRVNK

The amino acid sequence of the N-terminally truncated modified recombinant collagenase fused to His-tag (HHHHHH) followed by a tobacco etch virus (TEV) cleavage site (NLYFO) at the N-terminus—are denoted by any one of SEQ ID NOs: 5-7 below:

Modified recombinant collagenase polypeptide
(also referred to as Des1)-
SEQ ID NO: 5
MGSSHHHHHHSSGENLYFQGGTMYDFEYLNGLSYTELTNLIKNIK
WNQINGLFQYSTGSQKFFGDKNRVQAIINALQESGRTYTANDDKG
IETFTEVLRAGFYLGYYYDGLSYLNDRNFQDKCIPAMIAIQKNPN
FKLGTAVQDEVITSLGKLIGNASANAEVVNNCVPVLKQFRENLNQ
YAPDYVKGTAVYELIKGIEYDESGAAYEKDVKTMPWYGKIDPFIN
ELKALGLYGNITSDTEWASDVGIYYLSKFGLYHTNRNDIVQSLEK
AVDMYKYGKIAFVAMERITWDYDGIGSNGKKVDHDKFLDDAEKHY
LPKTYTFDNGTFIIRAGDKVSEEKIKRLYWASREVKSQFHRVVGN
DKPLEVGNADDVLTMKIFNSPEEYKFNTNINGVSTDNGGLYIEPR
GTFYTYERTPQQSIFSLEELFRHEYTHYLQARYLVPGLWGQGPFY
EKNRLTWFDEGTAEFFAGSTRTSGVLPRKLILGYLAKDKVDHRYS
LKKTLNSGYDDSDWMFYNYGFAVAHYLYEKDMPTFIKMNKAILNN
DVKSYDEIIKKLSDDANKNTEYQNHIQELVDKYDNATIPLVSDDY
LKDHGYKKASEVYSEISKAANLTNTSVTEHKSQYENTFTLRGTYT
GETSKGEFKDWKEMSKKLDGTLESLAKNSWSGYKTLTAYFTNYRV
TSDNKVQYDVVFHGVLTDNGDISNNKAPIAKVTGPSTGAVGRNIE
FSGKDSKDEDGKIVSYDWDFGDGATSRGKNSVHAYKKAGTYNVTL
KVTDDKGATATESFTIEIKNEDTTTPITKEMEPNDDIKEANGPIV
EGVTVKGDLNGSDDADTFYFDVKEDGDVTIELPYSGSSNFTWLVY
KEGDDQNHIASGIDKNNSKVGTFKATKGRHYVFIYKHDSASNISY
SLNIKGLGNEKLKEKENNDSSDKATVIPNENTTMQGSLLGDDSRD
YYSFEVKEEGEVNIELDKKDEFGVTWTLHPESNINDRITYGQVDG
NKVSNKVKLRPGKYYLLVYKYSGSGNYELRVNK
Modified recombinant collagenase polypeptide
(also referred to as Des4)-
SEQ ID NO: 6
MGSSHHHHHHSSGENLYFQGGTMYDFEYLNGLSYTELINLIKNIK
WNQINGLFQYSDGSQKFYGDKNRVQAIINALQESGRTYTANDDKG
IETFTEVLRAGFYLGYYYDGLSYLNDRNFQDKCIPAMIAIQKNPN
FKLGTAVQDEVIASLGKLIGNASANAEVVNNCVPVLKQFRENLNQ
YAPDYVKGTAVYELIKGIEYDESGAAYEKDVKTMPWYGKIDPFIN
ELKKLGLYGNITSDTEWASDVGIYYLSKFGLYHTNRNDIVQALEK
AVDMYKYGKIAFVAMERIKWDYDGIGSNGKKVDHDKFLEDAEKHY
LPKTYTFDNGTFIIRAGDKVSEEKIKRLYWASKEVKAQFHRVVGN
DKPLEVGNADDVLTMKIYNSPEEYKFNTYINGVSTDNGGLYIEPR
GTFYTYERTPQQSIFSLEELFRHEYTHYLQARYLVPGLWGQGPFY
ENNRLTWFDEGTAEFFAGSTRTSGVLPRKTILGYLAKDKVDHRYS
LKKTLNSGYDDSDWMFYNYGFAVAHYLYEKDMPTFIKMHKAILNN
DVKSYDEIIKKLSDDANKNKEYQNHIQELVDRYDNATIPLVSDDY
LKDHGYKPASEVYSEISKAANLTNTSVTKHKSQFFNTFTLRGTYT
GGTSKGEFKDWKEMSKKLDETLESLSKKSWSGYKTLTAYFTNYRV
TSDNKVQYDVVFHGVLTDNGDISNNKAPIAKVTGPSTGAVGRNIE
FSGKDSKDEDGKIVSYDWDFGDGATSRGKNSVHAYKKAGTYNVTL
KVTDDKGATATESFTIEIKNEDTTTPITKEMEPNDDIKEANGPIV
EGVTVKGDLNGSDDADTFYFDVKEDGDVTIELPYSGSSNFTWLVY
KEGDDQNHIASGIDKNNSKVGTFKATKGRHYVFIYKHDSASNISY
SLNIKGLGNEKLKEKENNDSSDKATVIPNENTTMQGSLLGDDSRD
YYSFEVKEEGEVNIELDKKDEFGVTWTLHPESNINDRITYGQVDG
NKVSNKVKLRPGKYYLLVYKYSGSGNYELRVNK
Modified recombinant collagenase polypeptide
(also referred to as Des6)-
SEQ ID NO: 7
MGSSHHHHHHSSGENLYFQGGTMYDFEYLNGLSYDELTNLIKNIK
WNQINGLFQYSDGSQKFYGDKNRVQAIINALEESGRTYTANDDKG
IETFTEVLRAGFYLGYYYDGLSYLNDRNFQDKCIPAMIAIQKNPN
FKLGTDVQDEVIAALGKLIGNASANAEVVNNCVPVLKQFRENLNQ
YAPDYSKGTAVYELIKGIEYDFSGAAYEKDPKTMPWYGKIDPFIN
ELKKLGLYGNITSDTEWASNVGIYYLSKFGKYHSNRNDIVQALEK
AVDMYKYGKIAFVAMERIKEDYDGIGSNGKKVDHDKFKEDAEKHY
LPKTYTFDNGTFIIRAGDKVSEEKIKRLYWASKEVKAQFHRVVGN
DKPLEVGNADDVLTMKIYNSPEEYKFNTYINGVSTDNGGIYIEPR
GTFYTYERTPQQSIFSLEELFRHEFTHYLQARYLVPGLWGQGPFY
ENNRLTWFDEGTAEFFAGSTRTSGVLPRKTILGYLAKDKVDHRYS
LKKTLNSGYDDSDWMFYNYGFAVAHYLYEKDMPTFIKMHKAILNN
DVKSYDEYIKKLSDDANKNKEYQNHIQELVDRYDNATIPLVSDDY
LKDHGYKPASEIYSEIAKAANLTNTSVTKHKSQFFNTFTLRGTYT
GGTSKGEFQDWKEMNKKLDEILEQLSKKSWSGYKTLTAYFTNYRV
TSDNQVQYDVVFHGVLTDNGDISNNKAPIAKVTGPSTGAVGRNIE
FSGKDSKDEDGKIVSYDWDFGDGATSRGKNSVHAYKKAGTYNVTL
KVTDDKGATATESFTIEIKNEDTTTPITKEMEPNDDIKEANGPIV
EGVTVKGDLNGSDDADTFYFDVKEDGDVTIELPYSGSSNFTWLVY
KEGDDQNHIASGIDKNNSKVGTFKATKGRHYVFIYKHDSASNISY
SLNIKGLGNEKLKEKENNDSSDKATVIPNFNTTMQGSLLGDDSRD
YYSFEVKEEGEVNIELDKKDEFGVTWTLHPESNINDRITYGQVDG
NKVSNKVKLRPGKYYLLVYKYSGSGNYELRVNK

Des1 includes 15 amino acid replacements/substitutions (approx. 2.2% of the protein). Most of the replacements were positioned on the surface of the protein. Surface exposed hydrophobic (or less hydrophilic) amino acids were mutated/substituted to more hydrophilic or even charged residues, raising solubility and thermal resistance, e.g. F295Y, A334D, S353H, T635N, Q669D, G670N, G672T, S701N and A709E. The latter can form a hydrogen bond with Y693 from an adjacent α helix. Moreover, the N203Y mutation can form pi-pi stacking with Tyr150 from a neighboring loop. Lastly, introducing prolines rigidify backbone stability, therefore A458P and D536P were introduced as well. (pssm=position-specific scoring matrix).

The mutations introduced in Des1 sequence are listed in Table 2:

TABLE 2
lists the mutations in the_amino acid sequence of thermo-stable
Des1 as denoted by SEQ ID NO: 2 relative to the amino acid sequence
of the full-length WT collagenase as denoted by SEQ ID NO: 8

| Position | WT | Des1 | |
|---|---|---|---|
| 334 | A | D | 1^{st} pssm surface polarity H bond internal bb |
| 458 | A | P | 1^{st} pssm loop rigidity |
| 709 | A | E | 1^{st} pssm with K. H bond with Y693 |
| 536 | D | P | 1^{st} pssm.loop rigidity |
| 737 | D | K | 1^{st} pssm |
| 710 | E | H | 1^{st} pssm H bond with N419. (nitrogen)) H bond |
| | | | network H710-N419-E709(bb) |
| 295 | F | Y | 1^{st} pssm.surface polarity |
| 670 | G | N | 1^{st} pssm.surface polarity |
| 672 | G | T | 1^{st} pssm.surface polarity H bond with bb of I673 |
| 183 | M | D | 1^{st} pssm.surface polarity H bond with internal |
| | | | bb D183 and bb of G185 |
| 149 | N | Q | 1^{st} pssm.surface flexibility and stability |
| 203 | N | Y | 3^{rd} pssm pi stacking |
| 287 | N | Y | 1^{st} pssm. H bond with Lys 291 also surface |
| | | | polarity |
| 669 | Q | D | 1^{st} pssm surface polarity |
| 353 | S | H | 1^{st} pssm.surface polarity |
| 701 | S | N | 1^{st} pssm.surface polarity |
| 635 | T | N | 1^{st} pssm.surface polarity |

Expression and purification of modified recombinant collagenase G—modified collagenases with N-terminally truncated recombinant collagenase polypeptide (ColG) fused to His-tag (HHHHHH) followed by a tobacco etch virus (TEV) cleavage site (NLYFO) at the N-terminus—denoted by any one of SEQ ID NOs: 5-7-were cloned into a pET15b plasmid and transformed into E. coli competent cells. Selected colonies were transferred to LB medium and COLG protein expression is induced using 1 mM IPTG for 16 h at 25° C. Next, cells are harvested, and the modified collagenases and recombinant forms thereof were purified using immobilized metal affinity chromatography (IMC), followed by ion exchange chromatography (IEC).

In vivo experimental set-up—split-mouth design. Rats are being divided into several groups (TBD), and are acclimatized before the start of the experiment. On the day of the orthodontic procedure, or at the time of measuring tooth movement, the rats are being anesthetized using a mixture of 25 mg/kg body weight of ketamine HCl and 42 mg/kg body weight of xylazine hydrochloride by intramuscular injection (IM).

Orthodontic procedure—An ordinary orthodontic Ni—Ti closed coil spring (9 mm closed coil spring nickel and titanium alloy that has two eyelets with an inner radius of 0.76 mm) is being used to connect each first molar in the upper pallet to the front upper incisors of the rat. The Ni-Ti coil spring will be glued to the tooth (3 M UNITEK), generating a constant force of 200 G (1.96 N) when extended between 12 and 24 mm. This type of coil has been used and studied in orthodontics for years and has proven to be effective in orthodontic procedures. The procedure of installing the Ni—Ti coil closed spring is being performed using human orthodontics equipment and materials. The first upper molar and the upper incisors is being dried and cleaned using cotton swabs to remove any debris that accumulates. The teeth are being conditioned using Assure plus primer conditioning agent by Reliance Orthodontics for 5-10 s creating a rugged surface to allow for stronger bonding. Following the conditioning, a small amount of composite Transbond LR light-cure adhesive (3 M UNITEK) is being spread over the molar. The eyelet ring of the Ni—Ti closed coil spring is being placed in parallel with the tooth and light cured for 10-15 s. Once again, a small amount of bonding agent is being spread over the ring and light cured for at least 40 s. The binding of the incisors is being performed in a similar manner. Cleaning, drying, and conditioning of the incisors is being done initially. Subsequently, a stainless ligature is being placed through the second eyelet ring of the Ni—Ti closed coil spring, and strong binding achieve by braiding it around the incisors. Illustration of the NITI closed coil that is being glued to the incisor and the occlusal surface of the first molar is presented in FIG. 8.

Injection of recombinant ColG and recombinant modified collagenase to the lamina propria of the marginal gingiva—As shown in FIG. 2 (Right side) Collagenase or PBS are being injected into the lamina propria of the marginal gingiva using a computer-assisted injection system (Wand Single Tooth Anesthesia system, Milestone Scientific, Roseland, NJ, USA), which enabled controllable and precise injection through a clinically accepted approach. Standard cartridges containing the local anesthetic solution for dental injection are accurately emptied of their content, washed with PBS and filled with either collagenase or PBS solution. Injection is being performed using a 30 G 2.54 cm length needle inserted into the marginal gingiva. The injection is being repeated at four sites around the molars: on the buccal, lingual, mesial, and distal sites, in a total volume of 50 μl. Collagenase are hydrated or dialyzed in PBS and used at a range of concentrations starting from conc. of about 0.20 mg/ml or about 0.50 mg/ml, and including concentrations of about 1.0 mg/ml, or about 1.2 mg/ml, or about 2.0 mg/ml, and including conc. of about 4 mg/ml, or more, in order to evaluate dose response, according to some embodiment.

Fiberotomy procedure—Circumferential fiberotomy is being made using a 11C scalpel inserted between the free gingiva and the tooth until reaching the bone level.

Measurement procedure—measurements of OTM are being performed and post-orthodontic relapse is also being measured. Data is being collected using intra-oral scanner (TRIOS 5 from 3Shape, Copenhagen, Denmark), and calculations are being performed by comparing deviations on the superimposed 3D intraoral scans of the maxilla, using Geomagic Control X software (Geomagic U.S., Research Triangle Park, NC), in a similar manner to that described in the measurement procedure in Example 1. Exact days of measurements and the exact calculations TBD.

### Example 4—N-Terminally Truncated Modified Recombinant (Des1) Collagenase has Thermo-Stable Characteristic

Thermal stability of the modified truncated recombinant collagenase protein was evaluated.—A heat inactivation assay was performed to test for thermal stability of the recombinant proteins by preincubating the purified proteins-truncated un-modified recombinant collagenase (ColG) (SEQ ID NO: 1) and the modified recombinant collagenase (Des1) (SEQ ID NO: 5), at temperatures ranging between 35 and 90° C. for 1 h. Residual activity was then measured by monitoring collagenase activity (as described in Tohar R. et. al., Int. J. Mol. Sci. 2021, 22, 8552).

The midpoint of temperature inactivation, the temperature at which 50% of the activity was retained (or lost) (T50), was determined by fitting a two-state model using GraphPad.

The results presented in FIG. 6, demonstrate that the T50 of the N-terminally truncated recombinant collagenase (ColG) was 53.6° C., whereas the T50 of the modified recombinant collagenase (Des1) was 56.3° C., in some embodiments.

The results clearly demonstrate that the recombinant modified collagenase was more thermally stable as compared to the recombinant truncated un-modified collagenase, and hence exhibited improved physical and biochemical properties.

### Example 5—In Vitro Activity Assay of the Isolated COLG Protein and Commercial Collagenase (C.COL)

In-vitro activity assay was performed to test the activity of the purified protein (C) with respect to that of a commercial collagenase (C.COL) (Sigma-Aldrich). In addition, to test whether the activity and readout were specific to collagen degradation by COLG and C.COL, each of the reactants and reagents from the biochemical reaction was depleted.

Briefly, following the protein purification process and dialysis of the collagenases into the activity assay's buffer, 10 μl aliquots (1.5 mg/ml) were transferred into a new 96-well plate in triplicates and incubated with 80 μg/ml collagen, followed by the addition of the reaction reagents.

FIG. 7 shows the fluorescence intensity resulting from the reaction under the various tested conditions. No significant fluorescence readouts were observed in wells without DHPAA or collagenase. On the other hand, the wells that did not contain collagen showed a significant difference between the purified protein (COLG) and the commercial one (C.COL), suggesting the basal florescence of the latter is higher without any collagen degradation related activity (p<0.05). Wells without collagenases had a significantly lower fluorescence intensity (FLI) than those with collagenases (p<0.0001). Finaly, where all the reaction components were included (two most right bars) significantly higher fluorescence was detected. This shows the specificity of the assay for collagen degradation by COLG and C.COL.

Importantly, FIG. 7 shows that the activity of C.COL was significantly higher, increase of about 30%-40%, than that of COLG (p<0.0001) (two most right bars), in some embodiments.

### Example 6—Comparison of the Effect of Enzymatic Fiberotomy and Traditional Surgical Fiberotomy on the Rate of Orthodontic Tooth Movement and Post-Orthodontic Relapse

Generally, the experiment involves administering the recombinant purified Collagenase (WT, truncated, and modified) into rats' marginal gingiva to impart enzymatically driven fiberotomy. The rate of orthodontic tooth movement following traditional and enzymatic fiberotomy is compared in vivo based on a model of a 0.012 nickel-titanium wire compressed between the rats' first molars. Four different animal groups are tested in a split-mouth study where one side of the mouth receives either the traditional or enzymatic fiberotomy and the other serves as a control. Teeth movement is monitored for 14 days while force is applied, followed by additional 7 days without applying external force to evaluate post-treatment relapse.

### Materials and Methods

### In-Vitro Collagenase Preparation:

Transformation, expression and purification: Three clostridial collagenase isoforms plasmids expressing WT collagenase from Clostridium histolyticum, truncated Collagenase (ColG) and one or more modified forms of the truncated Collagenase, as well as plasmids expressing homologous collagenase isoforms from Clostridium tetani, including WT and truncated forms of a collagenase Clostridium tetani, are transformed and expressed in competent cells (E. coli BL21). Purification of the enzymes is performed using Ni2+ HisTrap FF column.

Activity assay: the purified enzymes and their combinations are incubated with collagen-I and checked for activity using a fluorescence intensity activity assay.

The study is performed on rats, since they have been shown to be the most suitable animals for the investigation of orthodontic tooth movement (OTM), and the anatomical structures on which tooth movement is based. Moreover, in comparison to humans, tooth movement occurs in rats at a much faster pace due to their increased metabolism which allows short duration experiments.

Study design: 24 female Sprague Dawley rats, 4-month old, 200-250 gram weight are divided into four groups:
Group A: 6 rats, split-mouth design, an enzymatic fiberotomy is performed on the first molar of one side while the contralateral side receives PBS injection as a control (PBS);
Group B: 6 rats, split-mouth design, a traditional fiberotomy using a scalpel are performed on the first molar of one side while the contralateral side serves as a control (PBS);
Group C: 6 rats, spilt-mouth design, an enzymatic fiberotomy is performed on the first molar of one side and traditional fiberotomy is performed on the contralateral side.
Group D: 6 rats, spilt-mouth design, to check whether the injection per se provokes an effect, PBS is injected on the first molar of one side and the contralateral side serves as a negative control.

Study design: Case crossover (split mouth) design is applied, where randomly assigned one side of the mouth receives the traditional fiberotomy or the enzymatic one and the other side receives phosphate-buffered saline (PBS) that serve as a control.

Orthodontic procedure: The appliance is designed to apply an equal orthodontic movement on both sides of the upper jaw by a 0.012 nickel-titanium (NITI) wire (3M Unitek, Monrovia, CA 91016, USA) connected to small orthodontic tubes bonded to the mesial side of the first molars with Transbond™ XT (3M Unitek, Monrovia, CA 91016, USA). The experimental setting is shown in FIG. 1.

After the appliance is placed, the orthodontic force is applied for 14 days, followed by 7 days without force to observe and measure relapse.

### Administration of Collagenase:

Collagenase (WT, truncated and/or modified), or PBS are injected into the lamina propria of the marginal gingiva, using a computer-assisted injection system (Wand Single Tooth Anesthesia system, Milestone Scientific, Roseland, NJ, USA) by one investigator (O.C), which enables a controllable and precise injection through a clinically accepted approach. The injection is repeated at four sites around the molars, on the buccal, lingual, mesial and distal at a total volume of 100 μl. Concentration of the selected collagenase/s is about 5.8 mg/ml.

Fiberotomy procedure: Circumferential fiberotomy is performed using a scalpel up to the alveolar bone level.

### Measurement Procedure:

Analysis of OTM (day 0,7) and post-orthodontic relapse (day 21) with associated bone microarchitectural changes is addressed (blinded to the treatment groups) using microcomputed tomography (μCT), at a duplicate manner at least 14 days apart. The measurement takes place on a 2D slice through the height of contour of the molars. Minimal distances are measured between the palatal suture and the first molars contour. This measurement method is used to evaluate OTM.

statistics: The mean and standard deviation (SD) of first molar tooth movements were statistically analyzed for changes with time, within the same group or between groups, using one-way ANOVA. Statistical significance was considered when p<0.05.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

1.-44. (canceled)

45. A method of performing an orthodontic procedure in a subject in need thereof, the method comprising administering to the subject in need thereof an effective amount of a recombinant collagenase polypeptide, or a composition comprising the same.

46. The method of claim 45, wherein the orthodontic procedure comprises one or more procedures selected from accelerated orthodontic tooth movement (OTM), post-orthodontic relapse prevention and teeth alignment, or any combination thereof.

47. The method according to claim 45, wherein the recombinant collagenase comprises one or more collagenases selected from: a full-length WT collagenase, N-terminally truncated un-modified collagenase, and N-terminally truncated modified collagenase, or any combination thereof.

48. The method according to claim 45, wherein the recombinant collagenase comprises an amino acid sequence having at least about 65% sequence identity to an amino acid sequence of a reference collagenase; wherein the reference collagenase comprises one or more collagenases selected from: a full-length WT collagenase having an amino acid sequence as denoted by SEQ ID NO:8, an N-terminal truncated collagenase having amino acid sequence as denoted by SEQ ID NO: 1 or SEQ ID NO: 9, N-terminally truncated modified collagenase having amino acid sequence as denoted by any one of SEQ ID NOs: 2-7; or any combination thereof.

49. The method according to claim 48, wherein the collagenase comprises an amino acid sequence having at least about 65% sequence identity to an amino acid sequence of the N-terminally truncated reference collagenase having amino acid sequence as denoted by SEQ ID NO: 1 or SEQ ID NO: 9.

50. The method according to claim 45, wherein the collagenase originates from Clostridium H (histolyticum) and/or Clostridium T (Tetani).

51. The method according to claim 45, wherein the collagenase polypeptide has an amino acid sequence having at least about 80% sequence similarity to an amino acid sequence as denoted by SEQ ID NO: 1 and/or SEQ ID NO: 9.

52. The method according to claim 45, wherein the collagenase polypeptide has an amino acid sequence as denoted by SEQ ID NO: 1 and/or SEQ ID NO: 9.

53. The method according to claim 45, wherein the collagenase polypeptide comprises an amino acid sequence having at least 80% similarity to an amino acid sequence denoted by any one of SEQ ID NO: 2, SEQ ID NO: 3, SEQ ID NO: 4, SEQ ID NO: 5, SEQ ID NO: 6, SEQ ID NO: 7, or any combination thereof.

54. The method according to claim 45, wherein the collagenase polypeptide comprises an amino acid sequence as denoted by any one of SEQ ID NOs: 1-9.

55. The method according to claim 45, wherein the collagenase polypeptide comprises an amino acid sequence as denoted by SEQ ID NO: 2 ad/or SEQ ID NO: 5.

56. The method according to claim 45, wherein the administration is local administration.

57. The method according to claim 45, wherein the administration is by injection.

58. The method according to claim 45, wherein the administration is to a connective tissue of marginal gingiva (lamina propria).

59. The method according to claim 45, wherein the recombinant collagenase polypeptide or the composition comprising the same are capable of affecting gingival fibers.

60. The method according to claim 45, wherein the recombinant collagenase polypeptide is administered at an effective amount of at least about 1.2 mg/ml.

61. The method according to claim 45, wherein the recombinant collagenase polypeptide is administered at an effective amount of at least about 4 mg/ml.

62. The method according to claim 45 wherein the orthodontic procedure utilizing the recombinant collagenase polypeptide or the composition comprising the same, provides equal or enhanced effect on tooth movement (mm), compared to same orthodontic procedure utilizing surgical fiberotomy; and wherein the orthodontic procedure comprises accelerated orthodontic tooth movement (OTM).

63. The method according to claim 45, wherein the orthodontic procedure utilizing the recombinant collagenase polypeptide or the composition comprising the same, provides equal or improved reduction in relapse compared to relapse after same orthodontic procedure utilizing surgical fiberotomy; and wherein the orthodontic procedure comprises prevention of post-orthodontic relapse.


SEQUENCE LISTING (9 sequences)

SEQ ID NO: 1 - protein, 1023 aa, synthetic construct
MGSSHHHHHHSSGENLYFQGGTMYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKLILGYLAKDKVDHRYSLKKTLNSGYDDSDWMFYNYGFAVAHYLYEKDMPTFIKMNKAILNTDVKSYDEIIKKLSDDANKNTEYQNHIQELVDKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 2 - protein, 1000 aa, synthetic construct
YDFEYLNGLSYTELTNLIKNIKWNQINGLFQYSTGSQKFFGDKNRVQAIINALQESGRTYTANDDKGIETFTEVLRAGFYLGYYYDGLSYLNDRNFQDKCIPAMIAIQKNPNFKLGTAVQDEVITSLGKLIGNASANAEVVNNCVPVLKQFRENLNQYAPDYVKGTAVYELIKGIEYDFSGAAYEKDVKTMPWYGKIDPFINELKALGLYGNITSDTEWASDVGIYYLSKFGLYHTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKPLEVGNADDVLTMKIFNSPEEYKFNTNINGVSTDNGGLYIEPRGTFYTYERTPQQSIFSLEELFRHEYTHYLQARYLVPGLWGQGPFYEKNRLTWFDEGTAEFFAGSTRTSGVLPRKLILGYLAKDKVDHRYSLKKTLNSGYDDSDWMFYNYGFAVAHYLYEKDMPTFIKMNKAILNNDVKSYDEIIKKLSDDANKNTEYQNHIQELVDKYDNATIPLVSDDYLKDHGYKKASEVYSEISKAANLTNTSVTEHKSQYFNTFTLRGTYTGETSKGEFKDWKEMSKKLDGTLESLAKNSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 3 - protein, 1000 aa, synthetic construct
YDFEYLNGLSYTELTNLIKNIKWNQINGLFQYSDGSQKFYGDKNRVQAIINALQESGRTYTANDDKGIETFTEVLRAGFYLGYYYDGLSYLNDRNFQDKCIPAMIAIQKNPNFKLGTAVQDEVIASLGKLIGNASANAEVVNNCVPVLKQFRENLNQYAPDYVKGTAVYELIKGIEYDFSGAAYEKDVKTMPWYGKIDPFINELKKLGLYGNITSDTEWASDVGIYYLSKFGLYHTNRNDIVQALEKAVDMYKYGKIAFVAMERIKWDYDGIGSNGKKVDHDKFLEDAEKHYLPKTYTFDNGTFIIRAGDKVSEEKIKRLYWASKEVKAQFHRVVGNDKPLEVGNADDVLTMKIYNSPEEYKFNTYINGVSTDNGGLYIEPRGTFYTYERTPQQSIFSLEELFRHEYTHYLQARYLVPGLWGQGPFYENNRLTWFDEGTAEFFAGSTRTSGVLPRKTILGYLAKDKVDHRYSLKKTLNSGYDDSDWMFYNYGFAVAHYLYEKDMPTFIKMHKAILNNDVKSYDEIIKKLSDDANKNKEYQNHIQELVDRYDNATIPLVSDDYLKDHGYKPASEVYSEISKAANLTNTSVTKHKSQFFNTFTLRGTYTGGTSKGEFKDWKEMSKKLDETLESLSKKSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 4 - protein, 1000 aa, synthetic construct
YDFEYLNGLSYDELTNLIKNIKWNQINGLFQYSDGSQKFYGDKNRVQAIINALEESGRTYTANDDKGIETFTEVLRAGFYLGYYYDGLSYLNDRNFQDKCIPAMIAIQKNPNFKLGTDVQDEVIAALGKLIGNASANAEVVNNCVPVLKQFRENLNQYAPDYSKGTAVYELIKGIEYDFSGAAYEKDPKTMPWYGKIDPFINELKKLGLYGNITSDTEWASNVGIYYLSKFGKYHSNRNDIVQALEKAVDMYKYGKIAFVAMERIKEDYDGIGSNGKKVDHDKFKEDAEKHYLPKTYTFDNGTFIIRAGDKVSEEKIKRLYWASKEVKAQFHRVVGNDKPLEVGNADDVLTMKIYNSPEEYKFNTYINGVSTDNGGIYIEPRGTFYTYERTPQQSIFSLEELFRHEFTHYLQARYLVPGLWGQGPFYENNRLTWFDEGTAEFFAGSTRTSGVLPRKTILGYLAKDKVDHRYSLKKTLNSGYDDSDWMFYNYGFAVAHYLYEKDMPTFIKMHKAILNNDVKSYDEYIKKLSDDANKNKEYQNHIQELVDRYDNATIPLVSDDYLKDHGYKPASEIYSEIAKAANLTNTSVTKHKSQFFNTFTLRGTYTGGTSKGEFQDWKEMNKKLDEILEQLSKKSWSGYKTLTAYFTNYRVTSDNQVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 5 - protein, 1023 aa, synthetic construct
MGSSHHHHHHSSGENLYFQGGTMYDFEYLNGLSYTELTNLIKNIKWNQINGLFQYSTGSQKFFGDKNRVQAIINALQESGRTYTANDDKGIETFTEVLRAGFYLGYYYDGLSYLNDRNFQDKCIPAMIAIQKNPNFKLGTAVQDEVITSLGKLIGNASANAEVVNNCVPVLKQFRENLNQYAPDYVKGTAVYELIKGIEYDFSGAAYEKDVKTMPWYGKIDPFINELKALGLYGNITSDTEWASDVGIYYLSKFGLYHTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKPLEVGNADDVLTMKIFNSPEEYKFNTNINGVSTDNGGLYIEPRGTFYTYERTPQQSIFSLEELFRHEYTHYLQARYLVPGLWGQGPFYEKNRLTWFDEGTAEFFAGSTRTSGVLPRKLILGYLAKDKVDHRYSLKKTLNSGYDDSDWMFYNYGFAVAHYLYEKDMPTFIKMNKAILNNDVKSYDEIIKKLSDDANKNTEYQNHIQELVDKYDNATIPLVSDDYLKDHGYKKASEVYSEISKAANLTNTSVTEHKSQYFNTFTLRGTYTGETSKGEFKDWKEMSKKLDGTLESLAKNSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 6 - protein, 1023 aa, synthetic construct
MGSSHHHHHHSSGENLYFQGGTMYDFEYLNGLSYTELTNLIKNIKWNQINGLFQYSDGSQKFYGDKNRVQAIINALQESGRTYTANDDKGIETFTEVLRAGFYLGYYYDGLSYLNDRNFQDKCIPAMIAIQKNPNFKLGTAVQDEVIASLGKLIGNASANAEVVNNCVPVLKQFRENLNQYAPDYVKGTAVYELIKGIEYDFSGAAYEKDVKTMPWYGKIDPFINELKKLGLYGNITSDTEWASDVGIYYLSKFGLYHTNRNDIVQALEKAVDMYKYGKIAFVAMERIKWDYDGIGSNGKKVDHDKFLEDAEKHYLPKTYTFDNGTFIIRAGDKVSEEKIKRLYWASKEVKAQFHRVVGNDKPLEVGNADDVLTMKIYNSPEEYKFNTYINGVSTDNGGLYIEPRGTFYTYERTPQQSIFSLEELFRHEYTHYLQARYLVPGLWGQGPFYENNRLTWFDEGTAEFFAGSTRTSGVLPRKTILGYLAKDKVDHRYSLKKTLNSGYDDSDWMFYNYGFAVAHYLYEKDMPTFIKMHKAILNNDVKSYDEIIKKLSDDANKNKEYQNHIQELVDRYDNATIPLVSDDYLKDHGYKPASEVYSEISKAANLTNTSVTKHKSQFFNTFTLRGTYTGGTSKGEFKDWKEMSKKLDETLESLSKKSWSGYKTLTAYFTNYRVTSDNKVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 7 - protein, 1023 aa, synthetic construct
MGSSHHHHHHSSGENLYFQGGTMYDFEYLNGLSYDELTNLIKNIKWNQINGLFQYSDGSQKFYGDKNRVQAIINALEESGRTYTANDDKGIETFTEVLRAGFYLGYYYDGLSYLNDRNFQDKCIPAMIAIQKNPNFKLGTDVQDEVIAALGKLIGNASANAEVVNNCVPVLKQFRENLNQYAPDYSKGTAVYELIKGIEYDFSGAAYEKDPKTMPWYGKIDPFINELKKLGLYGNITSDTEWASNVGIYYLSKFGKYHSNRNDIVQALEKAVDMYKYGKIAFVAMERIKEDYDGIGSNGKKVDHDKFKEDAEKHYLPKTYTFDNGTFIIRAGDKVSEEKIKRLYWASKEVKAQFHRVVGNDKPLEVGNADDVLTMKIYNSPEEYKFNTYINGVSTDNGGIYIEPRGTFYTYERTPQQSIFSLEELFRHEFTHYLQARYLVPGLWGQGPFYENNRLTWFDEGTAEFFAGSTRTSGVLPRKTILGYLAKDKVDHRYSLKKTLNSGYDDSDWMFYNYGFAVAHYLYEKDMPTFIKMHKAILNNDVKSYDEYIKKLSDDANKNKEYQNHIQELVDRYDNATIPLVSDDYLKDHGYKPASEIYSEIAKAANLTNTSVTKHKSQFFNTFTLRGTYTGGTSKGEFQDWKEMNKKLDEILEQLSKKSWSGYKTLTAYFTNYRVTSDNQVQYDVVFHGVLTDNGDISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKATKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 8 - protein, 1118 aa, Clostridium histolyticum
MKKNILKILMDSYSKESKIQTVRRVTSVSLLAVYLTMNTSSLVLAKPIENTNDTSIKNVEKLRNAPNEENSKKVEDSKNDKVEHVKNIEEAKVEQVAPEVKSKSTLRSASIANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKSTKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 9 - protein, 1000 aa, synthetic construct
YDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKSTKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK